(19) United States
(12) Patent Application Publication
(10) Pub. No.: US 2014/0060390 A1
(43) Pub. Date: Mar. 6, 2014

(54) COMPOSITION COMPRISING PORTLAND CEMENT FOR USE IN VERTEBROPLASTY

(71) Applicants: Michael P. Hofmann, Birmingham West Midlands (GB); Gareth Wynn-Jones, Birmingham West Midlands (GB); Richard M. Shelton, Birmingham West Midlands (GB)

(72) Inventors: Michael P. Hofmann, Birmingham West Midlands (GB); Gareth Wynn-Jones, Birmingham West Midlands (GB); Richard M. Shelton, Birmingham West Midlands (GB)

(73) Assignee: SPINEART SA, Meyrin (CH)

(21) Appl. No.: 14/110,213

(22) PCT Filed: Apr. 5, 2012
(86) PCT No.: PCT/GB2012/000321

§ 371 Date: Oct. 30, 2013

Related U.S. Application Data
(60) Provisional application No. 61/471,999, filed on Apr. 5, 2011.

(30) Foreign Application Priority Data
Jun. 10, 2011 (GB) 1109804.3

(51) Int. Cl. A61L 27/02

(52) U.S. Cl. CPC A61L 27/025
USPC 106/717; 106/713; 106/734; 106/728

(57) ABSTRACT

A vertebroplastic cementitious composition includes a solid phase including Portland cement and a biocompatible additive which is a plasticizer and a hardening accelerant, wherein the amount of additive is between about 1% and about 15% by weight relative to the Portland cement; and an aqueous liquid phase, wherein the ratio of the solid phase to the liquid phase is between about 3 g/ml and about 6 g/ml. Methods of forming such compositions, treatment methods using such compositions and cementitious implants including such compositions are also described.

### FIELD OF THE INVENTION

The invention relates to the field of cementitious compositions comprising Portland cement which are suitable for use in vertebroplasty.

### BACKGROUND TO THE INVENTION

The human vertebral column (spine), normally consists of twenty four separate vertebrae, together with five fused vertebrae that form the sacrum and four fused vertebrae that form the coccyx. Vertebral sizes increase down the spinal column, with the cervical vertebrae being the smallest and the lumbar vertebrae being the largest. The lumbar region supports the majority of the body load during movement (Teoh S H, Chui C K. Journal Mechanical Behavior of Biomedical Materials. 2008 April; 1(2):115-39).

The vertebral column has many functions. These include:
1. Protecting the spinal cord and spinal nerves;
2 Supporting the majority of the body weight;
3. Providing a partly rigid and flexible axis for the body and a pivot for the head;
4. Playing an essential role in locomotion (Moor L, Dalley F. Clinical oriented anatomy. Lippincott Williams and Wilkins; 2006 and Middleditch A, Oliver J. Functional anatomy of the spine. Elsevier; 2005).

A typical vertebra consists of, a vertebral body, a vertebral (neural) arch and seven processes. The vertebral body is the anterior part of the vertebra that gives strength to the vertebral column and provides support for body weight (Middleditch A, Oliver J. Functional anatomy of the spine. Elsevier; 2005). The inner cores of the vertebral bodies are composed of spongy trabecular or cancellous bone and are surrounded by dense, hard cortical bone (Teoh S H, Chui C K. Journal Mechanical Behavior of Biomedical Materials. 2008 April; 1(2):115-39). This design is lighter than purely solid bone and is better for coping with dynamic loads as it is more flexible (Bogduk N. Clinical anatomy of the lumbar spine and sacrum. Elsevier; 2003). Between the vertebral bodies are inter-vertebral discs that are largely responsible for transferring load between vertebral bodies (Middleditch A, Oliver J. Functional anatomy of the spine. Elsevier; 2005).

There are many causes of vertebral compression fractures (VCFs) these include; horizontal compression of the spine e.g. many sporting injuries, spinal myelomas and osteoporosis (Jensen M E, et al. American Journal of Neuroradiology. 1997; 18:1897-904 and Cortet B, et al. Revue du rhumatisme (English ed). 1997:177-83).

### Osteoporosis

Osteoporosis is defined as ‘A systemic skeletal disease characterised by low bone mass and micro-architectural deterioration of bone tissue causing (trabecular thinning), with a consequent increase in bone fragility and susceptibility to fracture risk’ (Kanis J A. Osteoporosis. Blackwell Science; 1994).

In developed countries, osteoporosis is the leading cause of VCFs. In the United States and Europe alone there are approximately 440,000 and 700,000 new cases of the disease reported respectively each year (Anagnostis P, et al. Osteoporosis International. 2009 February; 20(2):197-2078 and Akesson K, Adami, S. The year in osteoporosis Boca Ration 2004), with medical annual costs for these two areas totaling in excess of $1.1 billion dollars (Johnell O. European Spine Journal. 2003; 12:168-9).

With the rapidly aging population in developed countries, the cases of osteoporosis are expected to increase greatly, escalating the financial burden of treatment for developed economies (Rigg B, Melton L J. Bone. 1995; 17:505-11). Therefore, research into an effective, safe and efficacious treatment for VCFs has never been more important. The most widely used treatment for VCFs is percutaneous vertebroplasty (PVP), the procedure has changed little in over 20 years and still uses polymethylmethacrylate (PMMA) cement as the main vertebral body filler. This cement satisfactorily performs its PVP function, however, it also has many inadequacies and for improved treatments should ideally be revised or replaced.

### Percutaneous Vertebroplasty (PVP) and Kyphoplasty (KP)

PVP and KP are the main procedures for treatment of VCFs including those caused by osteoporotic vertebral compression fractures, spinal metastases and vertebral myelomas (Jensen M E, et al. American Journal of Neuroradiology. 1997; 18:1897-904 and Cortet B, et al. Revue du rhumatisme (English ed). 1997:177-83). Both PVP and KP are minimally invasive surgical procedures which stabilise the fractured vertebral body by injection of a bone cement material into the vertebra fracture (Taylor R S, et al. Spine. 2006 November; 31(23):2747-55). Bone cement is injected through a small hole in the skin (percutaneously) into a fractured vertebra with the goal of relieving the pain of osteoporotic compression fractures. During the procedure, bone cement is injected with a biopsy needle into the collapsed or fractured vertebra. The cement dries and forms a support structure within the vertebra that provides stabilization and strength. KP varies from normal PVP at the injection stage. Prior to injection of the cement a balloon is inflated in the vertebra in order to restore the vertebral body height and kyphotic angle of 8.5° (Lewis G. Journal of Biomedical Material Research Part B-Applied Biomaterials. 2006 February; 76B(2):456-68 and Heini P F, Orler R. European Spine Journal. 2004 May; 13(3):184-92). Evidence has suggested that this modified technique also reduces cement leakage which can lead to reduced cases of pulmonary embolism (Taylor R S, et al. European Spine Journal. 2007 August; 16(8):1085-100).

Vertebroplasty techniques are also reviewed extensively in Verlaan, J-J, et al Biomaterials, 27 (2006) 290-301. This paper also describes calcium phosphate cements for use in vertebroplasty. It is indicated that the stability after curing that Polymethyl methacrylate cement offers cannot be achieved with calcium phosphate cement.

### Disadvantages of the Present Bone Cement Polymethylmethacrylate (PMMA)

There are many procedural complications associated with PVP and its associated techniques. Some of these complications such as, haemorrhage, infection and increased risk of vertebral collapse (VC), are due to the surgical procedure and are independent of the type of bone cement used (Laredo J D, Hamze B. Skeletal Radiology. 2004 September; 33(9):493-505). The other main reason for complication is the use of the bone cement PMMA. Originally used due to its high compression strength and favourable setting times it has been associated with many side effects:
The polymerisation of methyl methacrylate is an exothermic process leading to possible necrosis of contacting tissues (Provenzano M J, et al. American Journal of Neuroradiology. 2004 August; 25(7):1286-90). Exothermic setting also prevents inclusion of sensitive biologically active ingredients such as growth factors or antibiotics through their denaturation at high temperatures. The same monomer has also shown to lead to hypersensitivity, allergic dermatitis and mucosal irritation (Darre E, et al. Pharmacology Toxicology. 1993; 72:332-5).
The PMMA cement monomer, through radiopacity monitoring, has been shown to leak from its site of injection in the vertebra in some cases. The methyl methacrylate monomer is toxic, has been shown to damage osteocytes (Dahl O E, et al. Acta Orthop Scand 1994; 65:147-53) and has been also linked to pulmonary embolism (Laredo J D, Hamze B. Skeletal Radiology. 2004 September; 33(9):493-505).
PMMA cement is a relatively inert material that does not resorb and thus may reside in the body for many years. This problem may pose little problem for elderly patients where the cement will probably have a clinical life span of approximately 20 years, but may be problematic for younger patients.

Portland cement is used in certain dental applications in the form of mineral trioxide aggregate (MTA) (U.S. Pat. No. 5,769,638). However, MTA is difficult to inject through a syringe and has a setting time of several hours. Additionally, MTA has a high material cost.

In view of the drawbacks of PMMA, and calcium phosphate cement, as a cement for vertebroplasty, the inventors have devised an alternative cement.

### SUMMARY OF THE INVENTION

In a first aspect, the invention provides a vertebroplastic cementitious composition comprising:
a solid phase comprising Portland cement and a biocompatible additive which is a plasticiser and a hardening accelerant, wherein the amount of additive is between about 1% and about 15% by weight relative to the Portland cement; and
an aqueous liquid phase,
   wherein the ratio of the solid phase to the liquid phase is between about 3 g/ml and about 6 g/ml.

The composition of the invention is cementitious in that it is capable of hardening and setting independently. It has been found that the cementitious composition can be injected through a hypodermic needle more easily and has a decreased setting time relative to unmodified Portland cement. Further, the composition hardens and sets without causing a significant increase in temperature, thereby avoiding possible necrosis and allowing the inclusion of biologically active ingredients. These properties make the composition suitable for use in vertebroplasty.

The composition is a vertebroplastic cementitious composition. This is a cementitious composition which is used in vertebroplasty. The terms “vertebroplastic” and “vertebroplasty” are also intended to cover kyphoplasty. Therefore, the cementitious composition of the invention can be used in kyphoplasty and can be considered to be a kyphoplastic cementitious composition

Portland cement is a hydraulic cement which hardens and sets after being combined with water. ASTM C150 standard specification for Portland cement defines Portland cement as a hydraulic cement produced by pulverizing clinker consisting essentially of hydraulic calcium silicates, usually containing one or more of the forms of calcium sulphate as an inter-ground addition.

To manufacture Portland cement, an intimate mixture of limestone and clay is ignited in a kiln to form Portland cement clinker. The following four main phases of Portland cement are present in the clinker-tricalcium silicate (3CaO.SiO₂, also referred to as C₃S), dicalcium silicate (2CaO.SiO₂, called C₂S), tricalcium aluminate (3CaO.Al₂O₃ or C₃A), and tetracalcium aluminoferrite (4CaO.Al₂O₃.Fe₂O₃ or C₄AF). The resulting clinker containing the above compounds is inter-ground with calcium sulphates to desired fineness to produce the Portland cement. Other compounds present in minor amounts in Portland cement include double salts of alkaline sulphates, calcium oxide, and magnesium oxide.

The term “Portland cement” is well known to one skilled in the art and is intended to cover all forms of Portland cement. This includes Type I, Type II, Type III, Type IV and Type V Portland cement as defined by ASTM C150. It also includes grey Portland cement, white Portland cement, and other forms of Portland cement. In one embodiment, the Portland cement used is grey Portland cement.

In embodiments of the invention, mineral trioxide aggregate (MTA) may be used as a source of Portland cement and/or as an alternative to Portland cement.

The solid phase should comprise at least about 40 wt % Portland cement. In various embodiments, the solid phase may comprise at least about 45 wt % Portland cement, at least about 50 wt % Portland cement, at least about 55 wt % Portland cement, at least about 60 wt % Portland cement, at least about 65 wt % Portland cement, at least about 70 wt % Portland cement, at least about 75 wt % Portland cement or at least about 80 wt % Portland cement.

The biocompatible additive can be any additive which is a plasticiser and a hardening accelerant. The additive, as a plasticiser, allows the cementitious composition to flow more easily, thereby allowing it to be extruded from a hypodermic needle in a greater quantity under a given force. When 5 ml of the cementitious composition is loaded into a 5 ml syringe and a maximum force of 100N is applied using an Instron 5544 universal testing machine, at least about 70% of the cementitious composition is preferably extruded from the syringe (for more details of this test, please see the Materials and Methods section in the detailed description of the invention). More preferably, at least about 75%, at least about 80%, at least about 85%, at least about 90%, at least about 95%, or about 100% of the cementitious composition is extruded from the syringe.

Further, the additive, as a hardening accelerant, causes the cementitious composition to harden and set more quickly than unmodified Portland cement. Preferably, the initial setting time of the cementitious composition is less than about 60 minutes as measured using the standard Gilmore needles test (Teoh S H, Chui C K. Bone material properties and fracture analysis: Needle insertion for spinal surgery. Journal Mechanical Behavior of Biomedical Materials. 2008 April; 1(2):115-39.) Preferably, the initial setting time of the cementitious composition is less than about 55 minutes, about 50 minutes, about 45 minutes, about 40 minutes, about 35 minutes, about 30 minutes, about 25 minutes or less than about 20 minutes. Preferably, the initial setting time of the cementitious composition is more than about 5 minutes. More preferably, the initial setting time of the cementitious composition is more than about 10 minutes. In particular embodiments, the initial setting time of the cementitious composition may be between about 10 minutes and about 30 minutes or between about 15 minutes and about 25 minutes.

When the cementitious composition sets, it should have sufficient compressive strength to allow it to support the vertebral column and the loads/compressive forces associated therewith. Preferably, the composition has a compressive strength after 1 day of at least about 50 MPa. More preferably, the composition has a compressive strength after 1 day of at least about 55 MPa, about 60 MPa, about 65 MPa, about 70MPa, about 75 MPa, about 80 MPa, about 85 MPa, or at least about 90 MPa. Compressive strength can be tested using the protocol described below.

Preferably, the composition has a compressive strength after 10 days of at least about 50 MPa. More preferably, the composition has a compressive strength after 10 days of at least about 55 MPa, about 60 MPa, about 65 MPa, about 70 MPa, about 75 MPa, about 80 MPa, about 85 MPa, or at least about 90 MPa.

The additive is a biocompatible additive. The term ‘biocompatible’ as used herein means that the additive to which the term refers is not unacceptably toxic, immunogenic, allergenic or pro-inflammatory when used in vivo in the composition of the invention.

Suitable biocompatible additives which are plasticisers and hardening accelerants are well known to those skilled in the art. The biocompatible additive can comprise one or more components. Where the biocompatible additive is one component, that component will act as both a plasticiser and a hardening accelerant. Where the additive is more than one component, for example, two components, one component may act as a plasticiser and the other may act as a hardening accelerant. Alternatively, where the additive is more than one component, for example, two components, both components may act as both a plasticiser and a hardening accelerant. Where the additive is more than one component, the components can be added to the Portland cement separately or as a mixture.

In preferred embodiments of the invention the biocompatible additive is a donor molecule, providing a source of ions. Preferably, the ions are chloride, nitrate, citrate or sulphate ions.

The biocompatible additive may be selected from one or more of the following: calcium chloride, calcium nitrate, sodium aluminate, sodium hexaphosphate, calcium acetate, citric acid, sodium citrate, calcium citrate and potassium citrate.

In particular embodiments, the additive may be the combination of calcium chloride and calcium nitrate. In other embodiments, the additive may be selected from citric acid, sodium citrate, calcium citrate and potassium citrate. In some embodiments, the additive may be sodium citrate or potassium citrate.

In the cementitious composition, the amount of additive relative to the Portland cement is between about 1% and about 15% by weight. For example, if the solid phase contains only Portland cement and additive at 10% by weight relative to the Portland cement, the solid phase will contain 90 wt % Portland cement and 10 wt % additive. Alternatively, if the solid phase contains 50 wt % filler, Portland cement and additive at 10 wt % by weight relative to the Portland cement, the solid phase will contain 50 wt % filler, 45 wt % Portland cement and 5 wt % additive.

In particular embodiments, the amount of additive relative to the Portland cement is between about 1% and about 12% by weight. In various embodiments, the amount of additive relative to the Portland cement may be between about 3% and about 12% by weight or between about 4% and about 11% by weight. In certain embodiments, the amount of additive relative to the Portland cement may be between about 3% and about 7% by weight, between about 4% and about 6% by weight, or be about 5% by weight. In other embodiments, the amount of additive relative to the Portland cement may be between about 8% and about 12% by weight, between about 9% and about 11% by weight or be about 10% by weight. In further embodiments, the amount of additive relative to the Portland cement may be between about 1% and about 3% by weight, between about 1.5% and about 2.5% by weight, or be about 2% by weight.

In particular embodiments in which the additive is selected from citric acid, sodium citrate, calcium citrate and potassium citrate, or the subgroups mentioned above, the amount of additive relative to the Portland cement may be between about 1% and about 5% by weight. In other embodiments, the amount of citrate additive relative to the Portland cement may be between about 1% and about 4% by weight, between about 1% and about 3% by weight, or between about 1.5% and about 2.5% by weight. In preferred embodiments of the invention the amount of citrate additive relative to the Portland cement is about 2% by weight.

The aqueous liquid phase can be any suitable aqueous liquid which can cause the solid phase of the cementitious composition to harden and set. The aqueous liquid may be water. Alternatively, the aqueous liquid can be an aqueous solution. For example, in some embodiments, the aqueous liquid phase may be an aqueous solution of a citrate salt such as sodium citrate or potassium citrate. The amount of citrate salt in solution may be between about 0.025% and about 0.1% by weight relative to the amount of Portland cement.

The ratio of the solid phase to the liquid phase is between about 3 g/ml and about 6 g/ml, i.e. between about 3 g and about 6 g of solid phase per ml of liquid phase. In some embodiments, the ratio of the solid phase to the liquid phase may be between about 4.5 g/ml and about 5.5 g/ml. In further embodiments, the ratio of the solid phase to the liquid phase may be between about 3.5 g/ml and about 4.5 g/ml. In yet further embodiments of the invention, the ratio of the solid phase to the liquid phase may be between about 2 g/ml and about 4.5 g/ml.

The solid phase may comprise further components. One such component is a radiopacifier or a radiopaque substance to allow the cementitious composition to be visualised after injection into the body. Suitable radiopacifiers can be in powder or liquid form. Powder radiopacifiers can be selected from one or more of bismuth oxide, barium sulphate, lanthanum oxide, zinc oxide, zirconium oxide, bismuth subnitrate, bismuth carbonate and tantalum oxide. Liquid radiopacifiers can be selected from one or more of 2-[2′,3′,5′-tribenzoyl]ethyl methacrylate (TIBMA) and 3,5-diiodine salicyclic methacrylate (DISMA).

In a particular embodiment of the invention, a vertebroplastic cementitious composition is provided, the composition comprising:
a solid phase comprising Portland cement, calcium chloride, and calcium nitrate, wherein the amount of each of the calcium chloride and the calcium nitrate is between about 2% and about 3.5% by weight relative to the Portland cement; and
an aqueous liquid phase,
   wherein the ratio of the solid phase to the liquid phase is between about 3.5 g/ml and about 4 g/ml.

Preferably, the amount of each of the calcium chloride and the calcium nitrate is between about 2.5% and about 3% by weight relative to the Portland cement.

In another embodiment of the invention, there is provided a vertebroplastic cementitious composition, the composition comprising:
a solid phase comprising Portland cement and a citrate salt, wherein the amount of citrate salt is between about 1.5% and about 2.5% by weight relative to the Portland cement; and
an aqueous liquid phase,
   wherein the ratio of the solid phase to the liquid phase is between about 4.5 g/ml and about 5.5 g/ml.

In this embodiment, the citrate salt is preferably sodium citrate or potassium citrate. More preferably, the citrate salt is sodium citrate. Preferably, the amount of citrate salt is between about 1.8% and about 2.2% by weight relative to the Portland cement. More preferably, the amount of citrate salt is about 2% by weight relative to the Portland cement.

Preferably, the ratio of the solid phase to the liquid phase is between about 4.8 g/ml and about 5.2 g/ml. More preferably, the ratio of the solid phase to the liquid phase is about 5 g/ml.

The solid phase of the cementitious composition may further comprise between about 8% and 12% bismuth oxide by weight relative to the Portland cement. Preferably, the solid phase of the cementitious composition further comprises between about 9% and 11% bismuth oxide by weight relative to the Portland cement. More preferably, the solid phase of the cementitious composition further comprises about 10% bismuth oxide by weight relative to the Portland cement.

The invention also provides a reactive cementitious powder comprising Portland cement and a biocompatible additive which is a plasticiser and a hardening accelerant, wherein the amount of additive is between about 1% and about 15% by weight relative to the Portland cement.

This powder can be mixed with an aqueous liquid at a powder to liquid ratio of between about 3 g/ml and about 6 g/ml to form the cementitious composition of the invention.

The invention further provides a reactive cementitious powder comprising Portland cement, calcium chloride, and calcium nitrate, wherein the amount of each of the calcium chloride and the calcium nitrate is between about 2% and about 3.5% by weight relative to the Portland cement.

This powder can be mixed with an aqueous liquid at a powder to liquid ratio of between about 3.5 g/ml and about 4 g/ml to form a cementitious composition.

Additionally, the invention provides a reactive cementitious powder comprising Portland cement and a citrate salt, wherein the amount of citrate salt is between about 1.5% and about 2.5% by weight relative to the Portland cement.

This powder can be mixed with an aqueous liquid at a powder to liquid ratio of between about 4.5 g/ml and about 5.5 g/ml to form a cementitious composition.

A skilled person will appreciate that the various limitations associated with the cementitious composition of the invention are applicable, where appropriate, to the reactive cementitious powders of the invention. For example, the solid phase of the cementitious composition is effectively equivalent to the reactive cementitious powder. Therefore, the amount of Portland cement present in the solid phase is also applicable to the reactive cementitious powder.

The reactive cementitious powder may comprise at least about 40 wt % Portland cement. In some embodiments, the reactive cementitious powder may comprise at least about 45 wt % Portland cement, at least about 50 wt % Portland cement, at least about 55 wt % Portland cement, at least about 60 wt % Portland cement, at least about 65 wt % Portland cement, at least about 70 wt % Portland cement, at least about 75 wt % Portland cement or at least about 80 wt % Portland cement.

In another aspect, the invention provides a preformed cementitious implant which is formed from the cementitious composition of the invention. The cementitious implant comprises a solid hydrated composition comprising Portland cement and a biocompatible additive which is a plasticiser and a hardening accelerant, wherein the amount of additive is between about 1% and about 15% by weight relative to the Portland cement.

The cementitious composition can be cast into a mould of a desired size and shape. The cementitious composition will harden and set in the shape of the mould. For example, cementitious implants may be in the form of blocks, granules, rods, sheets, sponges, pellets or other shapes. Blocks, once formed, may subsequently be pulverised to form granules, or their shape may be adapted to the intended use by fragmentation, abrasion or filing. The cement casts can be used as an implant for inserting into bone cavities and fractures. For example, the implant can be introduced into a bone cavity and additional unset cementitious composition can be added to the bone cavity around the implant.

The use of an implant resolves any biocompatibility issues that might occur due to the setting reaction.

In another aspect, the invention provides a method of forming a vertebroplastic cementitious composition, the method comprising:
mixing a solid phase comprising Portland cement and a biocompatible additive which is a plasticiser and a hardening accelerant, wherein the amount of additive is between about 1% and about 15% by weight relative to the Portland cement, with an aqueous liquid phase at a ratio of the solid phase to the liquid phase of between about 3 g/ml and about 6 g/ml to form a cementitious composition.

In one embodiment, the invention provides a method of forming a vertebroplastic cementitious composition, the method comprising:
mixing a solid phase comprising Portland cement, calcium chloride, and calcium nitrate, wherein the amount of each of the calcium chloride and the calcium nitrate is between about 2% and about 3.5% by weight relative to the Portland cement, with an aqueous liquid phase at a ratio of the solid phase to the liquid phase of between about 3.5 g/ml and about 4 g/ml to form a cementitious composition.

In another embodiment, the invention provides a method of forming a vertebroplastic cementitious composition, the method comprising:
mixing a solid phase comprising Portland cement and a citrate salt, wherein the amount of citrate salt is between about 1.5% and about 2.5% by weight relative to the Portland cement, with an aqueous liquid phase at a ratio of the solid phase to the liquid phase of between about 4.5 g/ml and about 5.5 g/ml to form a cementitious composition.

As above, a skilled person will appreciate that the various limitations associated with the cementitious composition of the invention are applicable, where appropriate, to the method of the invention.

The invention also provides a vertebroplastic cementitious composition for use in therapy, in particular, vertebroplasty and kyphoplasty.

Further, the invention provides a vertebroplastic cementitious composition for use in treating a condition associated with a bone cavity. The condition may be a vertebral compression fracture.

Also provided is a method of treatment comprising introducing a cementitious composition according to the invention into a bone cavity and allowing the cementitious composition to harden and set. Preferably, the composition is introduced by injection.

### BRIEF DESCRIPTION OF THE FIGURES

Cementitious compositions in accordance with the invention, and methods for their preparation and use, will now be described, by way of example only, with reference to the accompanying drawings, FIGS. 1 to 34 in which:

FIG. 1 shows cement extrusion of the PCS with calcium chloride, calcium nitrate and sodium citrate admixtures at 2 wt % at PLRs of 3.2, 3.6 and 4.0 g/ml.

FIG. 2 shows cement extrusion of the PCS with sodium aluminate, sodium hexaphosphate and calcium acetate at 2 wt % at PLRs of 3.2, 3.6 and 4.0 g/ml.

FIG. 3 shows the injectability of the PCS with calcium chloride, calcium nitrate and sodium citrate at 5 wt % at PLRs of 3.2, 3.6 and 4.0 g/ml.

FIG. 4 shows the injectability of the PCS with sodium aluminate, sodium hexaphosphate and calcium acetate at 2 wt % at PLRs of 3.2, 3.6 and 4.0 g/ml.

FIG. 5 shows the injectability of the PCS with 10 wt % of calcium chloride, calcium nitrate, sodium citrate, sodium aluminate, sodium hexaphosphate and calcium acetate.

FIG. 6 shows the force required to extrude PC containing 10 wt % calcium acetate which produced 97 wt % extrusion of the cement paste at a PLR of 4.0 g/ml (FIG. 5).

FIG. 7 shows the force required to extrude standard PC with 24.0 wt % cement paste extrusion (FIG. 6) at a PLR of 4.0 g/ml.

FIG. 8 shows a comparison of compounds containing the citrate anion on the injectability of PC.

FIG. 9 shows the results of investigating the effect on injectability of combining 2 or 5 wt % calcium chloride or calcium nitrate with 2 wt % sodium citrate at a PLR of 3.6 g/ml.

FIG. 10 shows a comparison of sodium and potassium citrate on the extrusion of PC at a PLR of 3.6 g/ml. (All admixtures were added in the powder phase).

FIG. 11 shows the injectability of the cement system with 5 wt % calcium chloride in combination with low quantities of sodium citrate. (PLR 4.0 g/ml, calcium chloride was added in the powder phase and sodium citrate in the liquid phase).

FIG. 12 shows the injectability of the cement system with 5 wt % calcium chloride in combination with low quantities of potassium citrate. (PLR 4.0 g/ml, calcium chloride was added in the powder phase and potassium citrate in the liquid phase).

FIG. 13 shows the initial setting times for the model PCS with the individual additions of 5 and 10 wt % of various admixtures.

FIG. 14 shows the compressive strength of the PCS with 5 wt % of calcium nitrate; sodium hexaphosphate, sodium aluminate, calcium chloride and calcium acetate at a PLR of 4.0 g/ml.

FIG. 15 shows the compressive strength of the PCS with 10 wt % of calcium nitrate, sodium hexaphosphate, sodium aluminate, calcium chloride and calcium acetate at a PLR of 4.0 g/ml.

FIG. 16 shows the relative porosities of the PCS with 5 wt % of various admixtures at a PLR of 4.0 g/ml.

FIG. 17 shows strut densities of the PCS with 5 wt % of various admixtures at a PLR of 4.0 g/ml.

FIG. 18 shows the injectability of PC with equal wt % of calcium chloride and calcium nitrate.

FIG. 19 shows the setting times of PC with equal wt % of calcium nitrate and calcium chloride.

FIG. 20 shows compressive strength values of 5 g of PC with equal quantities of calcium chloride and calcium nitrate.

FIG. 21 shows the relative porosities for the Portland cement system with equal quantities of calcium chloride and nitrate after 1 and 10-days setting at a PLR of 4.0 g/ml.

FIG. 22 shows strut densities for PC with equal quantities of calcium chloride and nitrate after 1 and 10-days setting at a PLR of 4.0 g/ml.

FIG. 23 shows an X-ray diffraction (XRD) pattern for (a) PC standard and (b) 5 wt % calcium chloride. The black line represents cements after 1-days setting and the grey line after 30-days setting.

FIG. 24 is an XRD pattern for (a) PC standard and 5 wt % of (b) calcium chloride (c) calcium nitrate (d) sodium hexaphosphate (e) calcium acetate and (f) sodium citrate. The black line corresponds to 1-day and grey line to 30-day cement setting.

FIG. 25 is an XRD pattern. The red line indicates the XRD pattern of the 2005 batch of PC and the black line indicates the 2010 batch of PC. Peak intensities were far higher in the new batch compared with the old batch.

FIG. 26 shows the injectability of PC with various liquefying agents.

FIG. 27 shows the injectability of PCs containing the radio-pacifier bismuth oxide.

FIG. 28 shows the initial setting times for PCs containing sodium citrate and bismuth oxide.

FIG. 29 shows the final setting times for PCs containing sodium citrate and bismuth oxide.

FIG. 30 shows the compressive strength of PC, PC with 10 wt % bismuth oxide, PC with 2 wt % sodium citrate and PC with 2 wt % citrate and 10 wt % bismuth oxide after 1-days setting. (All the cements were produced at a PLR of 5 g/ml).

FIG. 31 shows the relative porosities for PC, PC with 10 wt % bismuth oxide, PC with 2 wt % sodium citrate and PC with 2 wt % citrate with 10 wt % bismuth oxide after 1-days setting.

FIG. 32 is a SEM of PC containing 2 wt % sodium citrate after 1-days setting (a) 500× and (b) 2000× magnification and standard PC (c) 500×, (d) 2000× magnification.

FIG. 33 illustrates the growth curves obtained for 3T3 fibroblast cells (P5) incubated in Petri-dishes with PC standard and PC containing 5 wt % calcium chloride and nitrate. The cements were placed into the Petri-dishes at the same time as the cells.

FIG. 34 illustrates the growth curves obtained for 3T3 fibroblast cells (passage 5) (8) incubated in Petri-dishes with PC standard and PC containing 5 wt % calcium chloride and nitrate. The cements were placed into the Petri-dishes 24 hrs after the cells.

### DETAILED DESCRIPTION OF THE INVENTION

### Introduction

### Desirable Properties of Injectable Bone Cement for Use in Vertebroplasty

An injectable bone cement should possess:
A high monomer and cement radiopacity: As stated earlier the monomer can lead to acute physiological conditions when it leaks outside the vertebral body. Therefore, it is essential to be able to monitor both the cement and monomer post-operatively (Heine P F, et al. European Spine Journal. 2000; 9:445-50). Radiopaque substances such as tantalum sulphate, or zirconium dioxide have already been used as admixtures in PMMA. Cement extravasation is visualised and monitored using radiography (Provenzano M J, et al. American Journal of Neuroradiology. 2004 August; 25(7):1286-90).
Low curing temperatures: The curing temperature of PMMA commonly reaches approximately 50° C. depending on bulk size which may be linked to possible tissue necrosis (Belkoff S M, Molloy S. Spine Journal. 2003; 28:1555-9) so an isothermal setting temperature would be beneficial.
A working time of approximately 6-10 minutes and a setting time of 20 minutes (Lewis G. Journal of Biomedical Material Research Part B-Applied Biomaterials. 2006 February; 76B(2):456-68). Once the operation has finished the patient should be able to be discharged after 6 hours as this is normal for a PMMA PVP procedure.
The cement must easily mix with the liquid phase to reduce preparation time.
The paste should be injectable through a 2 mm surgeon's needle without the occurrence of blocking (Teoh S H, Chui C K. Journal Mechanical Behavior of Biomedical Materials. 2008 April; 1(2):115-39).
A high compression strength similar to PMMA of 79 MPa (Burton A W, et al. Neurosurgical Focus. 2005; 18(3):1-9).
Biocompatibility
No toxicity
Low cost.

### The Hydration of Portland Based Cement (PC)

Hydration of both the calcium silicates (alite and belite) leads to the creation of the calcium-silicate-hydrate phase (C—S—H) (Camilleri J, et al. Dental Biomaterials. 2005; 21:297-303), which provides the main strength of the PC paste. Alite reacts rapidly with water and forms long C—S—H bonds which harden the cement over the first 24 h. In contrast, belite reacts at a slower rate to form short C—S—H fibres and is more important for the long term strength of the cement. The tricalcium aluminate phase is one of the most reactive species forming C₄AF crystals on contact with water (Nonconventional concrete technologies: renewal of the highway infrastructure. National Research Council, National Materials Advisory Board, Comission of Engineering and Technical Systems; 1997. p. 14-24). These crystals are important for early setting and hardening of the cement paste.

### Properties of Portland Cement (PC) in Relation to its Use as PVP Cement

PC has many physical, chemical and biological properties that would be advantageous as a PVP cement.
High compressive strengths: are essential for cement supporting the vertebral column and the loads it will endure. PC has a compressive strength value of 72 MPa at a powder-to-liquid ratio of 4 g/ml after 10-days setting. This is comparative to the 65-100 MPa compression strength of PMMA (Gbureck U, et al. Biomaterials. 2004; 25(11):2187-95).
Aqueous setting: PC will set in an aqueous environment, this is crucial as extracellular fluid will be present in vivo.
Durability: PC has demonstrated long-term durability and stability, in the building industry, which is important for the intended clinical application (Yu H F, et al. Journal of Wuhan University of Technology-Materials Science Edition. 2008 December; 23(6):893-900).
Isothermal setting: Unlike PMMA, PC sets isothermally, therefore avoiding problems associated with tissue necrosis. It thus also allows for the inclusion of biologically active compounds such as antibodies, hormones and growth factors (Ber B S, et al. Journal of Endodontics. 2007 October; 33(10):1231-4).
Simple incorporation of radiopacifiers: As outlined earlier radiopacity is important for PVP cement in order to monitor cement leakage with radiography. Barium sulphate (BaSO₄), lanthanum oxide (La₂O₃) and tantalum pentoxide (Ta₂O₅) (Coomaraswamy K S, et al. 20th International Symposium on Ceramics in Medicine; 2007 Oct. 24-26; Nantes, FRANCE. Trans Tech Publications Ltd.) can all be used as radiopacifiers for PC.
Clinical history: PCs have been investigated for dental applications, hence only a clinical equivalency study would be required for PCs use in vertebroplasty (Camilleri J. International Endodontic Journal. 2008 December; 41(12):1107-14).
High alkalinity: PCs provide intrinsic antibacterial action but may also increases cell death (Camilleri J. International Endodontic Journal. 2008 September; 41(9):791-9).

### Challenges of Using PC as a Percutaneous Vertebroplasty (PVP) Material

There are several challenges that need to be overcome before PCs full potential can be realised as a PVP material. Firstly, aqueous cement systems, like PCs, are prone to phase separation, leading to in-homogeneity and weakening of the cement structure (Bohner M, et al. Journal of Materials Chemistry. 2008; 18(46):5669-75). The possibility of phase separation is increased during the 150 N pressure applied to the cement paste during injection.

Secondly, the mean setting time of the cement without additives is 3 to 4 hours (Ber B S, et al. Journal of Endodontics. 2007 October: 33(10):1231-4). As the vertebroplasty procedure requires approximately 20 minutes to complete the paste should remain fluid during the operation but set shortly afterwards (James L. Vertebroplasty & Kyphoplasty. Radiologyinfo.org; 2009 and Syed M I, Shaikh A. Pain Physician. 2007; 10(2):367-80).

The initial aims of the inventors were to firstly experiment with possible superplasticisers showing reduced phase separation during injection and to identify the candidates with the most potential. Secondly, PC accelerators were investigated for their effect on the setting time and compressive strengths of the Portland cement system (PCS). The admixture with the greatest superplasticising abilities were then combined with setting accelerators which produced cements with the highest compressive strengths.

### Materials and Methods

### Sample Preparation

Grey Portland Cement (PC) (Blue Circle Mastercrete, Lafarge, UK) was the main constituent of Portland Cement Systems (PCS) and was used as the control. Superplasticisers and hardening accelerators, grouped as admixtures, were added between 1-10 wt % into the powder phase of PC. These included calcium chloride, calcium nitrate, calcium acetate, sodium aluminate, sodium hexaphosphate, sodium citrate, calcium citrate and potassium citrate (Sigma, UK). Either distilled water or low concentrations of sodium or potassium citrate were used as the liquid phase for preparing the cement pastes (where a citrate based liquid phase was used it will be stated). Samples were always hand mixed for 1-1.5 minutes. The pastes were mixed at a range of powder-to-liquid ratios (PLRs) ranging between 3.2 to 5.5 g/ml.

### Testing Injectability of the PC Mixtures

After sample preparation the cement was loaded into a 5 ml syringe and tested for injectability using a customised jig for the Universal testing machine (Instron 5544).

The percentage of the cement mixture extruded from the syringe was calculated as follows:

Step 1: subtracting the syringe weight from the syringe and cement weight before the experiment to calculate the initial weight of the cement mixture before extrusion.
Step 2: performing the same calculation for the syringe after the experiment to calculate the final weight of the cement mixture after extrusion.
Step 3: equation 1 was used to calculate the wt % extrusion from the syringe.

$$( 1 - ( \frac{final wt of cement after extrusion ( g )}{intial wt of cement before extrusion ( g )} ) ) \qquad Equation 1$$

### Compressive Strength Testing

After sample preparation, the cement pastes were cast into cylindrical PTFE moulds (6 mm diameter, 12 mm height) and left to set for 6 h at 37° C. in a drying oven before being extracted and stored in distilled water at 37° C. for a further 10 days. After storage the wet samples were weighed and the dimensions measured with a digital Vernier caliper. The wet compressive strengths (CS) of the cement samples (n=10 per sample set) were then measured using a Universal testing machine (Inston 5544, UK) at a cross-head speed of 1 mm/min. CS values were then calculated using the following equation:

$$CS = \frac{L}{A} = \frac{4 L}{π \cdot {d}^{2}} \\ CS = Compressive strength ( MPa ) \\ L = Load ( N ) \\ A = cross \text{-} sectional area ( {mm}^{2} ) \\ d = diameter ( mm ) \qquad Equation 2$$

### Initial Setting Time Measurements

The initial setting times of the cements were measured under normal laboratory atmosphere (20-23° C. and 50-60% humidity) using the Gilmore needle test with a needle of 113.98 g and 2,117 mm diameter according to ASTM standard (ASTM-Standard. C266-99. ASTM International; 2002).

### Calculating the Dry Densities of the Cement Samples

Fragments obtained following the CS testing were collected, weighed and then air dried until the weight of the samples remained constant. The dry densities of the cement samples were then calculated using the following equation:

$${ρ}_{dry} = \frac{{W}_{D}}{{W}_{W}} \times {ρ}_{wet} \\ {ρ}_{dry} = Apparent dry density ( g \text{/} {cm}^{3} ) \\ {ρ}_{wet} = Apparent wet density ( g \text{/} {cm}^{3} ) \\ {W}_{D} = Dry weight of fragments ( g ) \\ {W}_{W} = Wet weight of fragments ( g ) \qquad Equation 3$$

### Calculating the Relative Porosities of the Cements

The dried cement samples obtained after drying the compression strength fragments were measured in a helium pycnometer (Accupyc 1330, Micromeritics, USA), which measured the materials strut densities. The relative porosities of the cement samples were then calculated using the following equation:

$$RP = 1 - \frac{{ρ}_{dry}}{{ρ}_{strut}} \\ {ρ}_{dry} = Apparent dry density ( g \text{/} {cm}^{3} ) \\ {ρ}_{strut} = Strut density ( g \text{/} {cm}^{3} ) \qquad Equation 4$$

### Scanning Electron Microscopy SEM Analysis.

Dried cement fragments were attached to an aluminium stub using silver nitrate paste before being gold coated (Emitech K550X). A JEOL 840A scanning electron microscope was then used to image the samples at an accelerating voltage of 11 kV using a stage height of 15 mm. Images were recorded at 500 and 2000× magnification.

### Energy Dispersive X-Ray Analysis.

An electron backscatter detector attached to an Oxford instruments 235 SEM was used to record the X-rays emitted by the elements on the surface of the cement, after irradiation with a beam of charged particles. Software (CMax) was then used to analyse the elemental composition. The software assigned both a weight % (wt %) and an atomic % for each element present. From the data the wt % ratio of each element pair was then calculated, and compared with theoretical compounds known to be present during cement hydration using the following calculation:

$$1 - ( \frac{Observed ratio - theoretical ratio}{observed ratio} ) * 100 \qquad Equation 5$$

### X-Ray Diffraction (XRD)

X-ray diffraction patterns of the set cements were recorded on a D8 Advance (Bruker, Germany). Data sets were collected from 2θ=5-40° with a step size of 0.02 and the count time was normalised to 1 s/step. The phase compositions of the cements were determined according to Inorganic crystal structure database, calcium hydroxide (PDF Ref. 04-010-3117), calcium silicate (PDF Ref. 04-011-1393) and ettringite (PDF 00-041-1451).

### Biological Testing

### Sample Preparation

PC powder was heat sterilised to 180° C. for 2 h and all the admixtures were added to solution and autoclaved. After sample preparation the hand mixed slurries were cast into polytetrafluoroethylene moulds, under aseptic conditions, producing cylindrical samples with 3 mm height and 6 mm diameter. The samples were then set for 6 h before being immersed in distilled water for 18 h and stored at 37° C. for 24 h.

### Cell Culture

### Preparation of Cell Culture Medium

Standard cell culture media comprised of the alpha modification of minimum essential medium (α-MEM) (Biosera, UK), supplemented with 1% penicillin/streptomycin (100 units/ml of penicillin with 100 μg/ml streptomycin (Sigma-Aldrich, UK), 2.5% HEPES buffer and 10% (v/v) foetal calf serum (FCS) (Biosera, UK).

### Cell Culture Preparation

A 3T3 fibroblast cell line (passage 5) was propagated in T-75 tissue culture flasks at 37° C., 5% CO₂ air until the flask were approximately 80-90% confluent. 4 ml of trypsin/EDTA 0.25% (w/v) was then used to detach the confluent monolayer. Once detached, an equal volume of supplemented α-MEM containing 10% FCS was added to neutralise the enzyme. The cell suspension was then collected and transferred into a 15 ml Falcon tube and centrifuged to pellet the cells. The supernatant was removed from the pellet, discarded and replaced with supplemented α-MEM containing 10% FCS, to resuspend the cells. To assess viable cell numbers, 50 μl of 0.4% trypan blue stain (Sigma-Aldrich, UK) was added to an equal volume of the resuspension. The contents were then mixed thoroughly and incubated for 5 minutes at room temperature. Following this, cells were observed using a phase contrast microscope (Zeiss, Germany) and live cells, where the trypan blue was unable to pass across the cell membrane, were counted and the cell concentration of the cell suspension was determined using a Neubauer haemocytometer.

### Cell Seeding, Monitoring and Feeding Schedules.

200 μl of the 3T3 cell line suspension was seeded at cell densities between 5-10×10⁴ cells ml⁻¹ in 35 mm culture dishes, cells were then either allowed to attach overnight before cements were placed in the centre of the well, or cement placement occurred at the same time as cell seeding. (Exact detail for each experiment will be given in the results section).

At 2-day intervals cells were trypsinised and viable cells counted using the trypan blue stain. Cells were fed at 3-day intervals using the cell culture media containing 10% FCS.

### Example 1

### Cements Containing Plasticisers and Hardening Accelerants

### Results

Investigating the Injectability of the PCS with 2, 5 and 10 wt % Admixture Additions

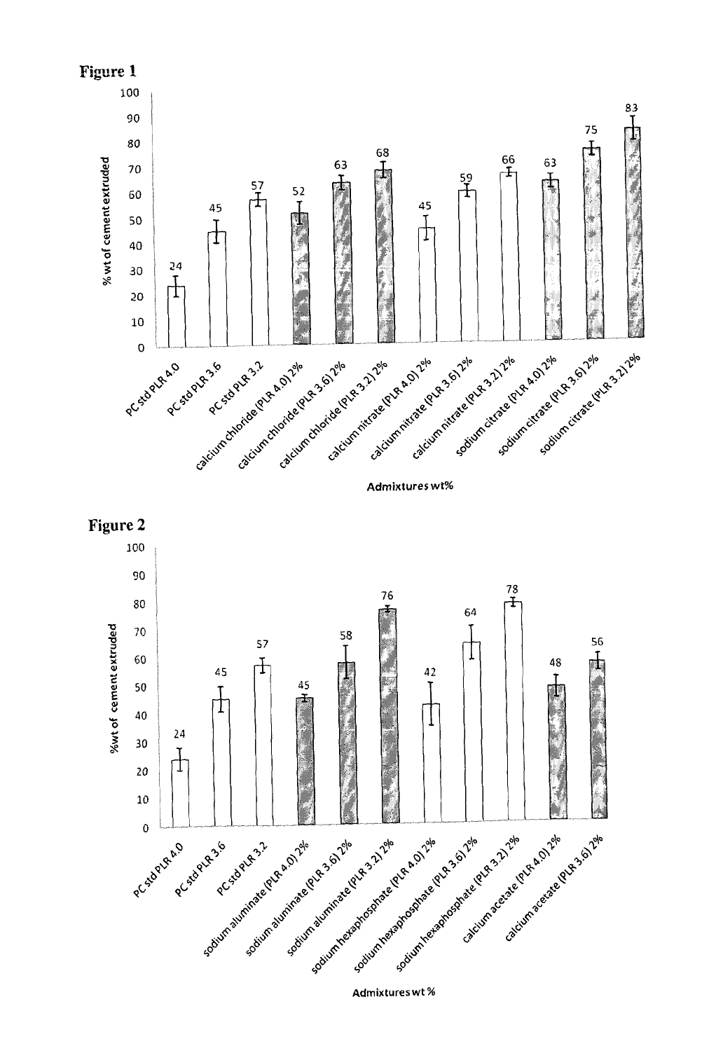

FIGS. 1 and 2 illustrate the effects on cement injectability of adding admixtures at 2 wt % to the PCS. For the PLR values of 3.2, 3.6 and 4.0 g/ml each of the admixtures added produced a significant increase in injectability compared with the PC standards (p<0.05). The most pronounced increase in cement extrusion was achieved with the addition of sodium citrate, which more than doubled extrusion compared with the PC standard at a PLR of 4.0 g/ml and produced over a 50% increase at a PLR of 3.2 g/ml.

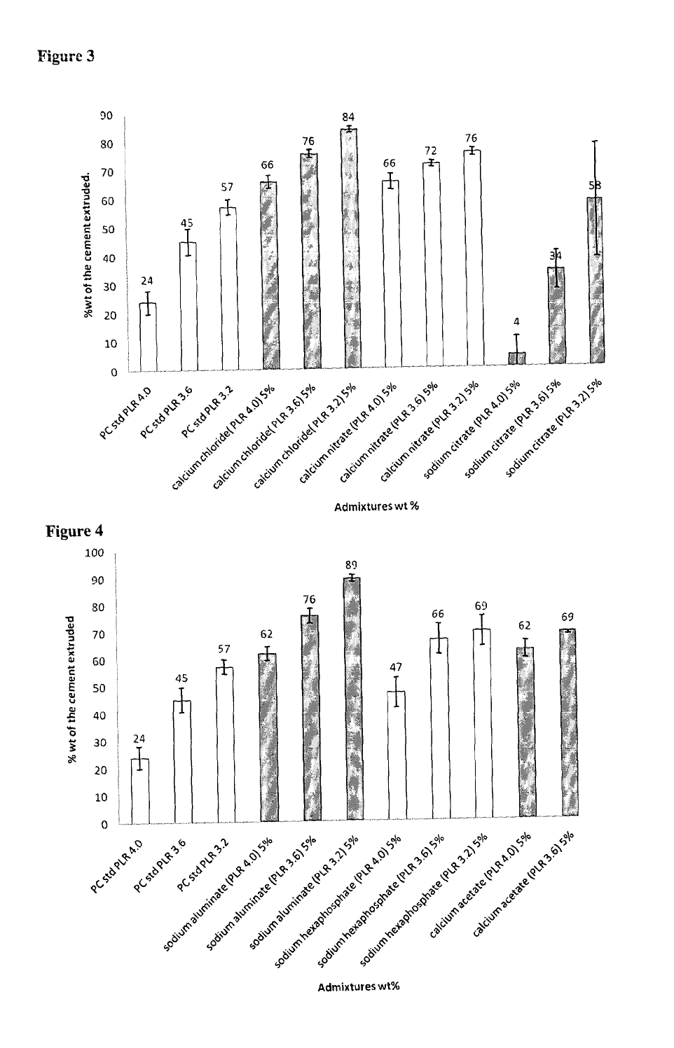

Increasing the admixture content from 2 to 5 wt % produced moderate increases in cement extrusion values for calcium chloride, calcium nitrate, sodium aluminate and calcium acetate (FIGS. 3 and 4). Sodium aluminate acted as the most powerful superplasticiser increasing extrusion by over 50% compared with the PC standard at 3.2 g/ml, generating extrusion values of nearly 90 wt %. In contrast, sodium citrate caused a decrease in extrusion values at all three of the PLR values tested. At a PLR of 4.0 g/ml inclusion of 5 wt % sodium citrate decreased extrusion by over 80% resulting in only 4 wt % cement extrusion. It was observed during the experiment that initially, cement mixtures produced with 5 wt % sodium citrate were very fluid, but then appeared to set during the process of transferring the cement to the syringe.

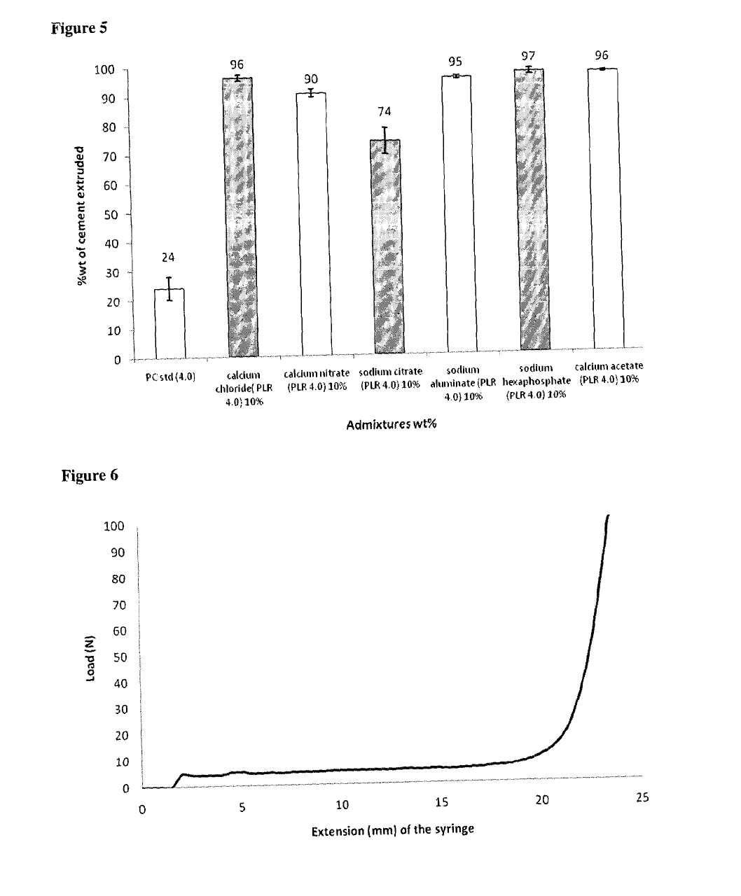

Doubling the quantity of all the superplasticisers from 5 to 10 wt % substantially increased cement extrusion (FIG. 5). Calcium chloride, sodium aluminate, sodium hexaphosphate and calcium acetate all produced cement extrusion values at a PLR of 4.0 g/ml of over 95 wt %, approximately 50% higher than the values for the 5 wt % admixture additions. Sodium citrate at 10 wt % restored injectability from the 5 wt % admixture additions to produce a cement extrusion value of over 70 wt %.

### Measuring the Load on the Syringe During the Injectability Experiments

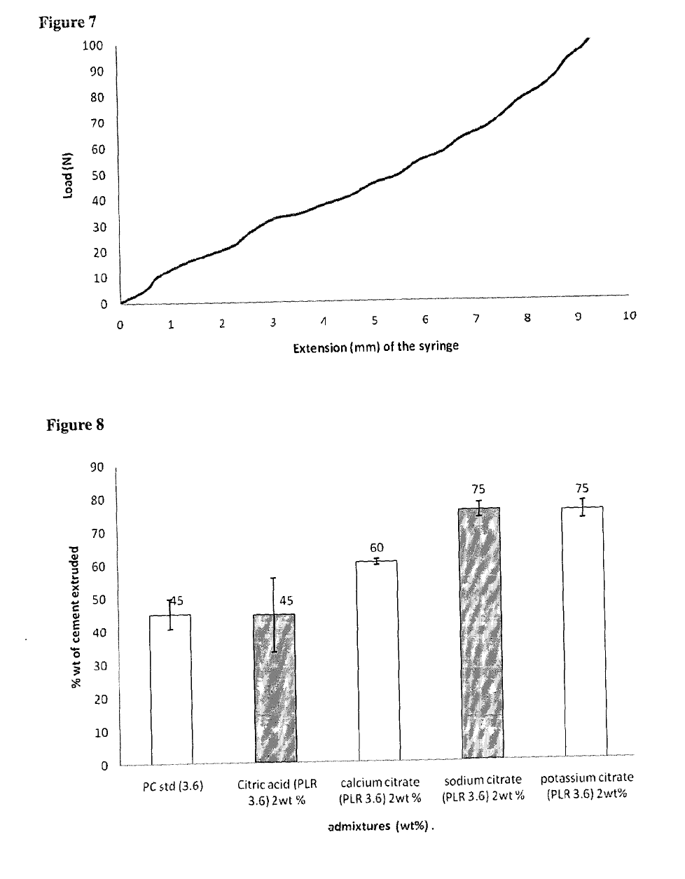

FIG. 6 demonstrates load (N) as a function of extension (mm) for a syringe containing the PCS with 10 wt % calcium acetate (for the cement extrusion values see FIG. 5). The graph was also typical for calcium chloride, calcium nitrate, sodium aluminate and sodium hexaphosphate modified cements at 10 wt %. A relatively low initial force e.g. 8 to 15 N was required to extrude the majority of the cement and an increase in force was observed only when the syringe was nearly empty. In contrast, FIG. 7 demonstrates the extension (mm) graph for a PC paste without any superplasticiser. There was no characteristic plateau of constant force but a continuous increase during the cement extrusion.

Investigating Injectability of the PCS with Compounds Containing the Citrate Anion

Additions of sodium and potassium citrate both produced over a 60% increase in cement extrusion compared with the PC standard at a PLR of 3.6 g/ml (FIG. 8). In contrast, citric acid additions generated had comparable extrusion values as the control and calcium citrate modified cements produced a moderate extrusion increase of 33%.

Investigating the Injectability of the PCS with Combinations of Either 5 wt % Calcium Chloride or Calcium Nitrate with 2 wt % Sodium Citrate

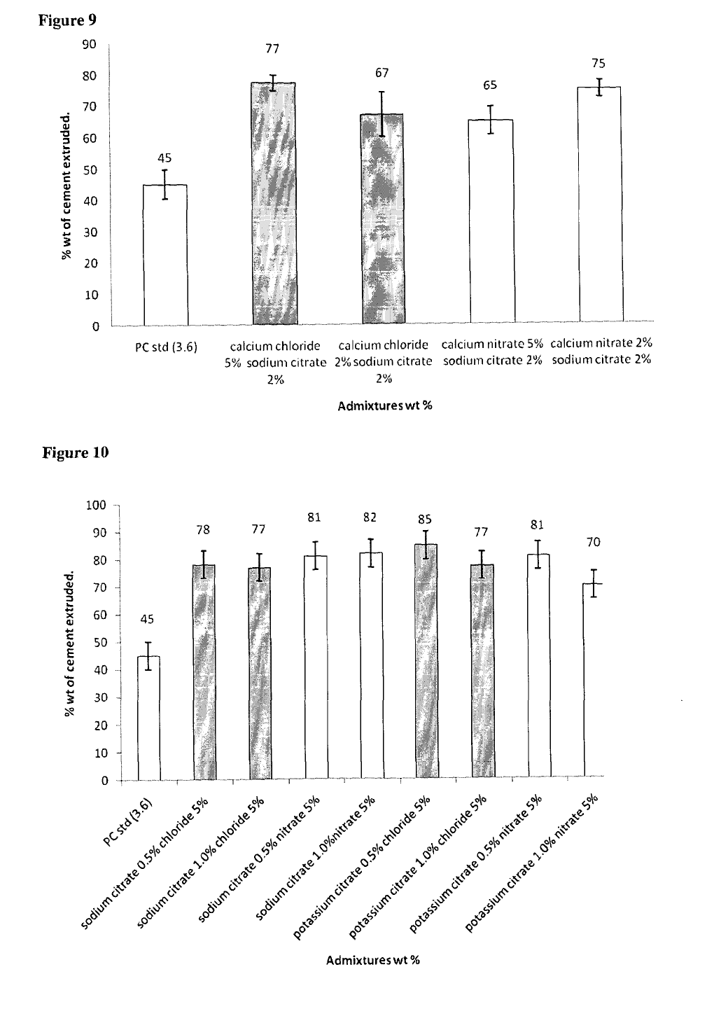

Calcium chloride or calcium nitrate additions at 2 or 5 wt % did not combine synergistically with sodium citrate at 2 wt % to produce cements with increased extrusion values compared with the individual admixtures (FIG. 9). For instance, 5 wt % calcium chloride addition produced a cement extrusion value of 76 wt % (FIG. 3) and 2 wt % additions of sodium citrate produced similar cement extrusion values of 75 wt % (FIG. 1). When calcium chloride and sodium citrate were combined at 5 and 2 wt % respectively the cements had a comparable extrusion value to using either admixture individually.

Investigating the Injectability of the PCS with Combinations of 5 wt % Calcium Chloride or Calcium Nitrate with Below 2 wt % of Either Sodium or Potassium Citrate

Decreasing the quantity of sodium citrate from 2 to 1 and 0.5 wt % produced negligible changes in cement extrusion (FIG. 10). Exchanging sodium for potassium citrate increased extrusion of the 0.5 wt % cements by 10% compared with the individual use of 5 wt % calcium chloride.

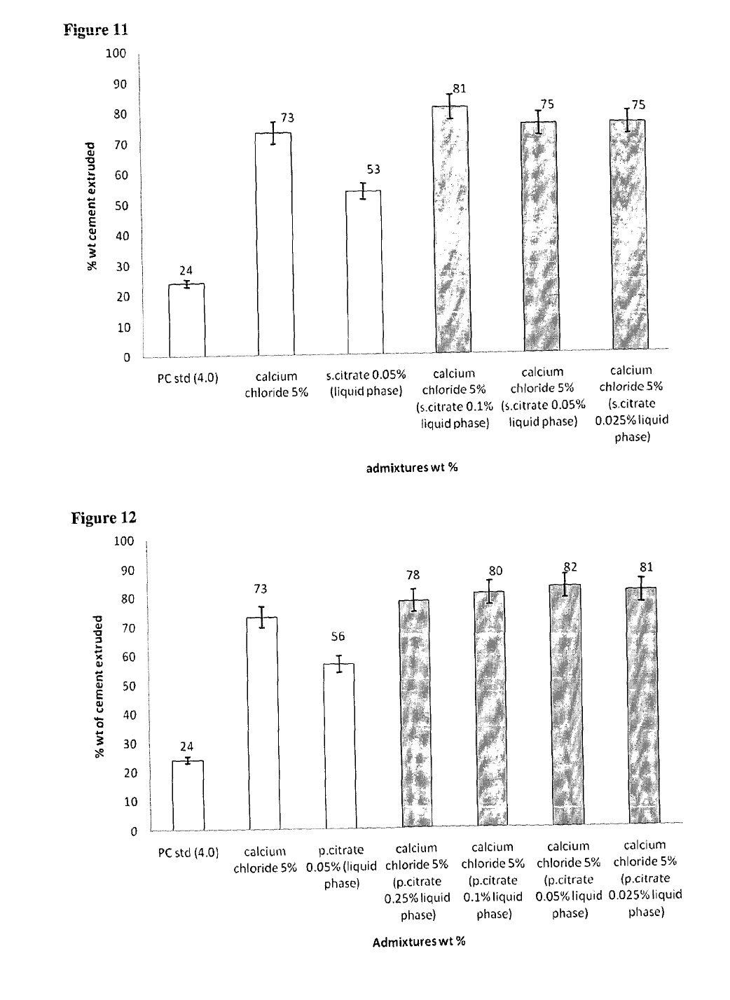

Further decreasing the sodium and potassium citrate addition from 0.5 to 0.025 wt % marginally decreased extrusion of the sodium citrate based cements, (FIG. 11), but did not affect extrusion of the potassium citrate based cements (FIG. 12). Individually, the additions of both sodium and potassium citrate increased extrusion by over double compared with the PC standard.

Investigating the Setting Times of the PCS with Individual 5 and 10 wt % Admixture Additions

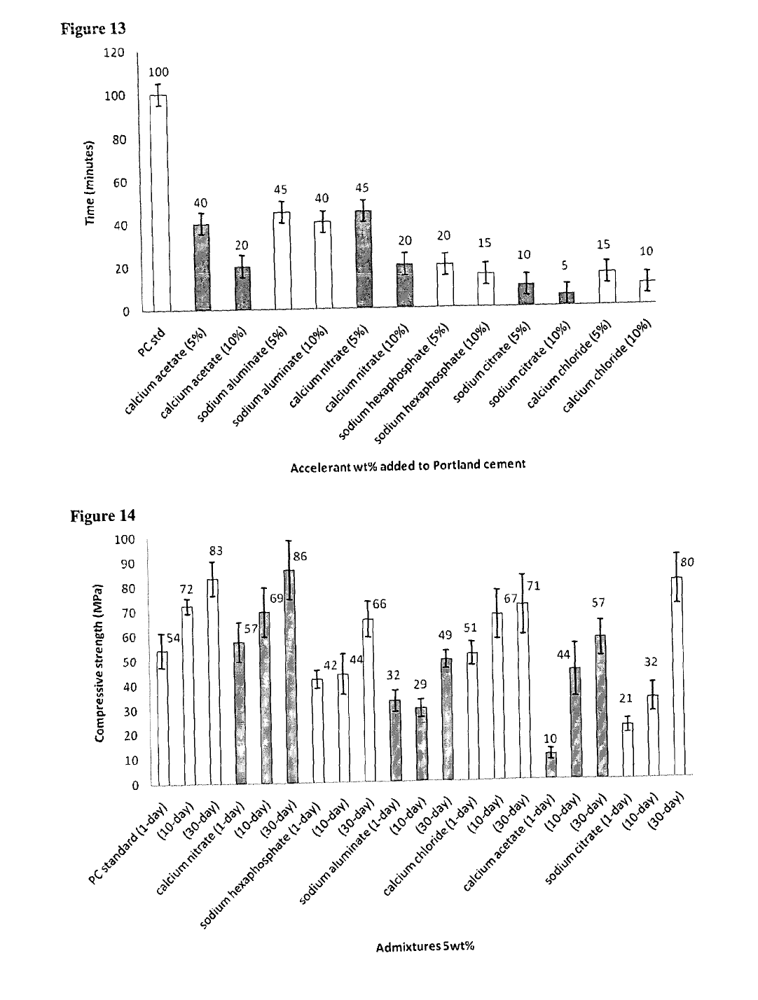

All of the admixtures added individually at 5 and 10 wt % at a PLR of 4.0 g/ml accelerated the setting of the cement to below 45 minutes, a significant reduction compared with the PC standard (p<0.05). All of the admixtures demonstrated reduced setting times when added at 10 wt % compared with 5 wt %. Sodium citrate was the most powerful accelerant setting at 10 wt % almost instantly. The majority of the superplasticiers at 10 wt % set in under 20 minutes including calcium acetate, sodium hexaphosphate and calcium chloride (FIG. 13).

Investigating the Setting Times of the PCS with Calcium Nitrate or Calcium Chloride Combined with Sodium or Potassium Citrate

Both sodium and potassium citrate at 0.5 and 2 wt % acted as significant cement retardents on PC setting (p<0.05) when combined with either 5 wt % calcium nitrate or calcium chloride (Table 1). The retarding effect of sodium and potassium citrate was further investigated by lowering the amount of the citrate based compounds to 0.025 wt % by adding them in the liquid phase (Table 2 and 3). Only cements containing less than 0.05 wt % sodium citrate, and 0.1 wt % potassium citrate, with 5 wt % calcium chloride set in under an hour.

TABLE 1
Demonstrating the retarding effect of sodium and potassium
citrate when combined with 5 wt % calcium chloride or
calcium nitrate (all admixtures were added in the powder phase).

| | Setting times | Setting time |
|---|---|---|
| | with 5 wt % | with 5 wt % |
| Admixture | calcium chloride | calcium nitrate |
| No citrate | 15 minutes ± | 45 minutes ± |
| | 5 minutes | 5 minutes |
| Sodium citrate 0.5 wt % | >6 h | >6 h |
| Sodium citrate 2 wt % | 5 h ± 30 minutes | >6 h |
| Potassium citrate 0.5 wt % | >6 h | >6 h |
| Potassium citrate 2 wt % | >6 h | >6 h |

The PC standard at a PLR of 4.0 g/ml set in approximately 100 minutes.

The PCS with 2 wt % sodium or potassium citrate>6 h setting times.

TABLE 2
Effect of further decreasing the wt % of sodium citrate on the
setting times of the PC mixtures. Only 0.05 and 0.025 wt %
sodium citrate additions produced setting times under 1 hour
(calcium chloride and nitrate were added in the powder phase,
sodium citrate in the liquid phase).

| | Setting times | Setting times |
|---|---|---|
| | with 5 wt % | with 5 wt % |
| Admixture | calcium chloride | calcium nitrates |
| Sodium citrate 0.025 wt % | 20 minutes ± | 1 h 30 minutes ± |
| | 5 minutes | 30 minutes |
| Sodium citrate 0.05 wt % | 35 minutes ± | 2 h 30 minutes ± |
| | 5 minutes | 30 minutes |
| Sodium citrate 0.1 wt % | 2 h ± 30 minutes | 4 h 30 minutes ± |
| | | 30 minutes |
| Sodium citrate 0.25 wt % | >6 h | 4 h 30 minutes ± |
| | | 30 minutes |
| Sodium citrate 0.5 wt % | >6 h | >6 h |
| Sodium citrate 2 wt % | >6 h | >6 h |

TABLE 3
Effect of further decreasing the wt % of potassium citrate on the
setting times of the PC mixtures. 0.025, 0.05 and 0.1 wt % all set
in under 5 minutes. (calcium chloride and nitrate were added in
the powder phase, sodium citrate was in the liquid phase.)

| | Setting times with 5 wt % |
|---|---|
| Admixture | calcium chloride |
| Potassium citrate 0.025 wt % | 12 minutes ± 5 minutes |
| Potassium citrate 0.05 wt % | 15 minutes ± 5 minutes |
| Potassium citrate 0.1 wt % | 35 minutes ± 5 minutes |
| Potassium citrate 0.25 wt % | 3 h ± 30 minutes |
| Potassium citrate 0.5 wt % | >6 h |
| Potassium citrate 2 wt % | >6 h |

Investigating the Compressive Strengths of the PCS with 5 and 10 wt % Admixture Additions after 1, 10 and 30 Days

The addition of 5 wt % calcium nitrate produced comparable compressive strength values to the 4.0 g/ml PC standard after 1, 10 and 30 days (FIG. 14). Calcium chloride also reduced compressive strength values compared with the standard cements after 1 and 10 days, but produced nearly 15% lower values after 30-days. In contrast, sodium hexaphosphate, sodium aluminate and calcium acetate each generated compressive strength values considerably lower than the PC standards. In particular, calcium acetates 1-day values were over 80% lower than the standard cements.

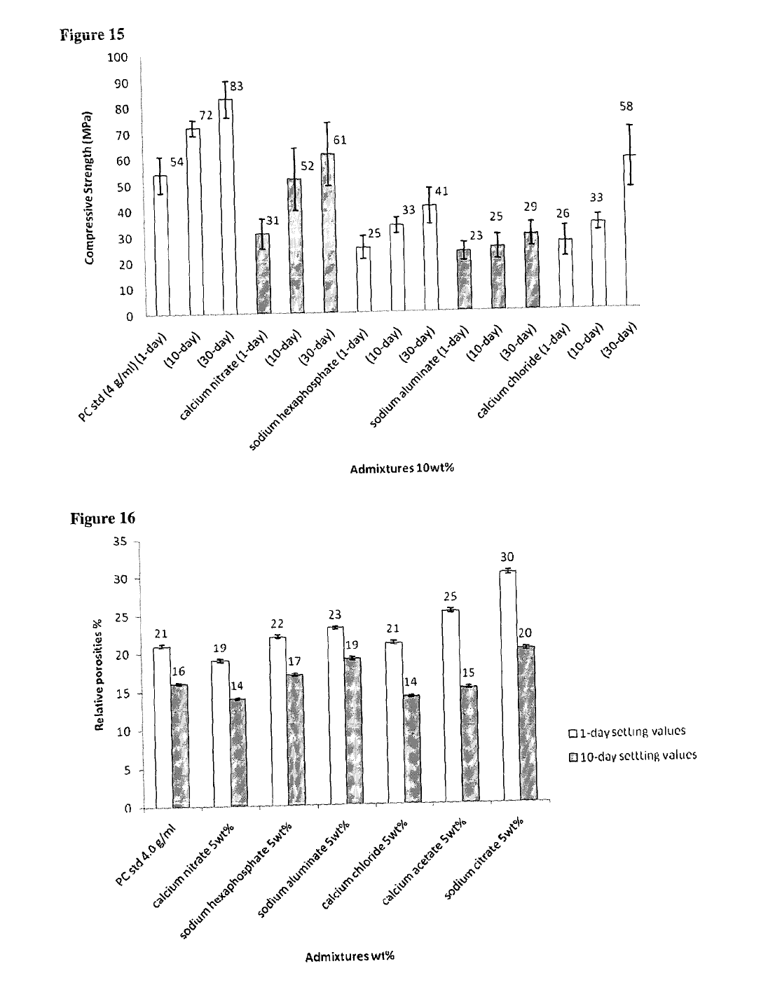

FIG. 15 demonstrates the effect of doubling the quantity of admixture from 5 to 10 wt % on the compressive strength values. None of the cements produced comparable strengths to the PC standards after 1, 10 or 30-days. Calcium nitrate again produced the highest compressive strength values but these were on average nearly 30% lower than the PC standard at all three time points. It was not possible to obtain 10 wt % compressive strength values for calcium acetate or sodium citrate. The samples produced with the former admixture were unstable and the latter set before the pastes could be transferred into the setting mould.

Investigating the Relative Porosities and Strut Densities of the PCS with 5 wt % Admixture Addition

Cements containing sodium citrate possessed the highest relative porosities by at least 5% points after 1-day cement setting (FIG. 16). In contrast, calcium chloride and nitrate additions produced the two lowest cement porosities after 10-days setting.

After 1-day of setting, cements containing sodium citrate possessed the highest strut densities by over 20% compared to the other modified cements (FIG. 17). In contrast, calcium nitrate and chloride led to the lowest values. After 10-days calcium acetate produced cements with the lowest strut densities. The most pronounced decrease in strut densities after 10-days setting was produced by sodium citrate.

### Discussion

Investigating Individual Admixture Additions with the PCS

### The Effect of 2, 5 and 10 wt % Admixture Additions on the Injectability of the PCS

Heat of hydration experiments have previously indicated that the citrate anion may prevent the dissolution of both the alite and aluminate phases of PC, both of which are essential for the evolution of early strength development in the cement paste (Moschner G, et al. Cement and Concrete Research. 2009 April; 39(4):275-82). Prevention of phase dissolution may explain the fluid consistency of the 2 wt % citrate cements which generated cement extrusion values of over 80 wt % (FIG. 1) but also the retarded cement setting times of the 2 wt % citrate modified cements (Table 1). Singh et al. (Singh N B, et al. Cement and Concrete Research. 1986; 16(6):911-20) utilised zeta-potential measurements to investigate the interaction of the citrate anion with the cement constituents. It was found that the drop in positive surface charge density of the set cement was an indication of the negative citrate anion binding to the positive cement surface. It has been suggested that binding results in repulsion between cement particles both electrostatically and sterically due to the large size of the citrate anion. This could disperse cement aggregates leading to a superplasticising effect, although it may also lead to the reduction in the rate of the cement setting reaction causing citrate to act as a retardant (Erdogdu S. Cement and Concrete Research. 2000; 30(5):767-73).

For other ceramic cements such as calcium phosphate cements (CPC), the superplasticising effects of citrate have been extensively investigated (Gbureck U, et al. Biomaterials. 2004; 25(11):2187-95 and Barralet J E, et al. Biomaterials. 2004; 25(11):2197-203). Gbureck et al (Gbureck U, et al. Biomaterials. 2004; 25(11):2187-95) investigated the injectability of the citrate based cements through a hypodermic needle for future use in clinical procedures such as vertebroplasty. The study concluded that citrate modified CPC cements generated over 95 wt % cement extrusion up to a PLR of 4.5 g/ml. Again, zeta-potential studies have suggested that the negative citrate anion acts by binding electrostatically to the positive surface of the cement (Gbureck U, et al. Biomaterials. 2004; 25(11):2187-95).

The second highest cement extrusion value for an admixture addition at 2 wt % was produced by sodium hexaphosphate. This admixture was another superplasticiser which has been previously studied with CPC. Zeta-potential measurements have also indicated that hexaphosphate interacts with the positively charged surfaces of the PC (Hesaraki S, et al. Journal of Biomedical Materials Research Part A. 2009; 88A(2):314-21). This indicates that possibly citrate and hexaphosphate share similar cement chemistry.

The other admixtures also demonstrated significant (p<0.05) increases in injectability compared with the PC standards at 2 wt % additions (FIGS. 1 and 2). Calcium acetate has previously demonstrated superplasticising abilities at a PLR of 3 g/ml at 2.5 wt % additions (El-Didamony H, et al. Ceramics-Silikaty. 1999; 43(1):29-33).

Increasing the admixture content from 2 to 5 and 10 wt % with the PCS produced a pronounced increase in extrusion values for the cements containing calcium chloride, calcium nitrate, sodium aluminate, calcium acetate and sodium hexaphosphate (FIGS. 3 to 5). Erdogdu (Erdogdu S. Cement and Concrete Research. 2000; 30(5):767-73) suggested that increasing the quantity of superplasticiser may lead to an increase in the electrostatic repulsion effect between the cement particles, leading to improved superplasticising abilities.

In contrast to the other admixtures, sodium citrate modified cements demonstrated a decrease in extrusion values when increasing the admixture from 2 to 5 wt % (FIG. 3). Although initially it appeared that during the injectability assessment the citrate based cement paste possessed a fluid consistency, observations suggested that the setting reaction was initiated before the mixture could be transferred to the syringe. Indeed, setting time measurements confirmed that sodium citrate with 5 wt % additions sets in less than 10 minutes (FIG. 13) which is in contrast with the 2 wt % citrate additions that required over 6 h to set. The setting reaction of citrate based PCs has previously been studied by calorimetrics and the final set cements characterised morphologically by scanning electron microscopy (SEM) (Ramachandran V S, Lowery M S. Thermochimica Acta. 1992; 195:373-87). These studies indicated that the final phase of the cement forms a weak non-crystalline calcium-silicate-hydrate (C—S—H phase) and a monosulphate phase which compares with the crystalline structure formed in the normal setting of PC.

### Measuring the Load on the Syringe During the Injectability Experiments

Authors studying the injectability of calcium phosphate cements obtained similar extension graphs for cements containing superplasticising admixtures with CPC cements (Gbureck U, et al. Biomaterials. 2004; 25(11):2187-95). The low initial force required to extrude the majority of the cement paste containing a superplasticiser suggested a homogeneous cement paste with few agglomerates (FIG. 6) (Habib M, et al. Acta Biomaterialia. 2008; 4(5):1465-71). In contrast, the continuous increase in force required to extrude the standard PC may be linked to the formation of high numbers of agglomerates in the paste (FIG. 7) The formation and frequency of agglomerates could be measured by cutting open the syringes after extrusion testing and performing a morphological SEM analysis of the cement paste remaining in the syringe, compared with the extruded cement paste.

### The Effect of 5 and 10 wt % Admixture Additions on the Setting Times and Compressive Strengths of the PCS

All of the admixtures at 5 and 10 wt % additions decreased the setting times of the cements compared with the PC standard, with the 10 wt % additions producing the shortest setting times (FIG. 13). Calcium chloride has previously been studied as a PC hardening accelerant in conjunction with samples containing bismuth oxide (Murphy J C, et al. Key engineering materials. 2008:1-9). In the study 5 wt % calcium chloride, decreased the setting time of the cement from 3 hours to 1.5 hours at a PLR of 4.0 g/ml. SEM examination of these cements indicated that in the presence of calcium chloride, more ‘interconnecting bridges’ were formed between the set cement particles than standard PCs. Another study inferred, through infra-red spectroscopy, that calcium chloride modified PCs demonstrated increased silicate polymerisation to produce a more structured C—S—H bond formation compared with standard PCs (Ber B S, et al. Journal of Endodontics. 2007; 33(10):1231-4). This structured bond formation possibly contributed to the early strength evolution of the PC samples containing 5 wt % calcium chloride in this study (FIG. 14). At 5 wt %, chloride modified cements possessed the second lowest relative porosities (FIG. 16) and low strut densities (FIG. 17) relative to cements containing other admixtures, the latter indicating that a high degree of conversion of the cement reactants occurred, suggesting a strong set structure had formed.

Calcium nitrate at 5 wt % produced a setting time of 45 minutes, which was comparatively long compared with the other admixtures tested (FIG. 13). However, 5 wt % calcium nitrate additions produced the highest compressive strength values of any admixtures after 1, 10 or 30-days of setting (FIG. 14). The nitrate based cements possessed low relative porosities and low strut densities, (FIGS. 16 and 17), the latter suggesting that the degree of hydration of cement reactant was higher than with other admixtures. The efficiency of the setting reaction for cements containing calcium nitrate has previously been linked to the belite (C₂S) content in the clinker phase (Aggoun S, et al. Construction and Building Materials. 2008 February; 22(2):106-10). Belite dissolves slowly to form short C—S—H fibres which provide long term strength for the cement (Chikh N, et al. Materials and Structures. 2008 January; 41(1):31-6) and the short C—S—H bonds possibly contributed to the high cement strengths for the nitrate modified cements.

Sodium aluminate, hexaphosphate, citrate and calcium acetate additions at 5 wt % all produced setting times under 25 minutes. However, each of these admixtures also reduced the compressive strength of the PCS (the only anomaly being the 30-day compressive strength values for sodium citrate) (FIG. 14). Sodium citrate based cements possessed the highest relative porosity of any cement sample after 1-day of setting (FIG. 16), indicating that a weak cement structure had been formed (Kendall K, et al. Philosophical Transactions of the Royal Society of London Series A, Mathematical and Physical Sciences. 1983; 310(1511):139-53). The admixture also produced the highest strut densities indicating a low degree of conversion of the cement reactants in the hydration reaction (FIG. 17). However, after 30-days sodium citrate produced similar compression strength values to the PC standards. This suggested that possibly the retarding effects of citrate were temporary and C—S—H bond formation began after 10 and before 30-days setting.

The 5 wt % calcium acetate based cements had the lowest strut densities of any admixture but higher than average porosities (FIGS. 16 and 17). This indicated that the degree of conversion of the cement reactants in the hydration reaction was high, resulting in a strong set structure (Kendall K, et al. Philosophical Transactions of the Royal Society of London Series A, Mathematical and Physical Sciences. 1983; 310(1511):139-53). However, high cement porosities led to the low compressive strength values possibly due to an increase in the critical flaw size of the set cement (FIG. 16). Calcium acetate addition up to 2.5 wt % has previously demonstrated strength increasing effects on PC (El-Didamony H, et al. Ceramics-Silikaty. 1999; 43(1):29-33).

Cement samples modified with sodium aluminate produced lower compression strength values than those modified with calcium chloride, nitrate or sodium hexaphosphate (FIG. 14). The influence of aluminate addition at 3.5 wt % to PC has previously been studied using NMR spectroscopy which indicated that aluminate accelerated the hydration of both the alite and belite phases, resulting in the creation of long C—S—H bonds (50). The porosities of the aluminate modified cements were average for the admixtures investigated but the strut densities were higher than average. The latter indicates that a low degree of hydration of the cement reactants had occurred, resulting in a weak strut structure. Furthermore, on hydration, an aluminate phase will set more quickly than other phases in the PC (Andersen M D, et al. Cement and Concrete Research. 2004 May; 34(5):857-68). This could have created a non homogeneous cement structure, resulting in cement flaws and consequently leading to the low compressive strengths.

### Investigating the Effect of Compounds Containing the Citrate Anion on the Injectability of the PC

As sodium citrate admixtures produced the highest PC extrusion values at 2 wt %, other compounds containing the citrate anion were studied for their effects on the PCS (FIG. 8). One explanation for the difference in cement extrusion values was the differing dissociation constants (K_{d}) of the various citrate based compounds. K_{d} is a measure of the reversible dissociation of an ionic compound in aqueous solution. The higher the K_{d} value, the greater the dissociation of the ions in solution. Equation 6 defines K_{d} for sodium citrate in solution.

Kd=([HOC(COO“−”)(CH2COO“−”)]+3[Nα“+”])/([HOC(COONα)(CH“2”COONα)“]”) Equation 6

Sodium citrate has a K_{d} value of 0.2 M and potassium citrate has a marginally higher value of 0.37 M (Mackenzie W. American Chemistry Journal. 1960; 65:159-61). In contrast, both calcium citrate and citric acid have K_{d} values lower than 1 mM. Therefore, there will be a higher concentration of the citrate anion in the cements containing sodium and potassium citrate than those containing calcium citrate or citric acid. The increased concentration of the superplasticising anion could explain the increased extrusion of cements containing sodium and potassium admixtures.

### Investigating the Effect of Combining Admixture Additions on the Injectability and Setting Times of the PCS

Sodium or potassium citrate at 2 wt %, the most powerful superplasticisers, were combined with calcium chloride or nitrate at 5 wt %, the two admixtures producing the highest compressive strength values. The admixtures did not combine synergistically to produce cements with increased cement extrusion values compared with those where the admixtures were added separately (FIG. 9). As the individual chloride and nitrate modified cements demonstrated setting times of under 10 minutes, it was conceivable that when the admixtures were combined, a premature setting was occurring preventing cement extrusion. However, setting times of the cements were all longer than 5 h, similar to the individual setting times of sodium and potassium citrate at 2 wt %, suggesting that the retarding effect of the citrate anion dominated the cement setting reaction (Table 1). In contrast, CPC containing sodium hydrogen phosphate as a setting accelerant set cements containing 1 to 3 wt % sodium citrate in 50 to 60 minutes which indicated that in the presence of certain accelerants setting in under an hour is possible for CPCs (Gbureck U, et al. Biomaterials. 2004; 25(11):2187-95).

The quantity of sodium and potassium citrate was then reduced below 2 wt % and combined with 5 wt % calcium chloride and nitrate before measuring setting times. The calcium chloride modified cements under 0.1 wt % were the only mixtures to set in under 2 hours. Calcium nitrate has been associated with the long term setting of PC which suggests that citrate prevented either the belite phase from dissolving or the strong C—S—H bonds from forming (Chikh N, et al. Materials and Structures. 2008 January; 41(1):3-6). In contrast, calcium chloride which was involved in the short term setting of PCs, may have formed the longer C—S—H fibres associated with short term setting (Singh N B, et al. Cement and Concrete Research. 2002 March; 32(3):387-92).

The moderate 10% increase in extrusion which was produced by potassium citrate modified cements with under 0.5 wt % citrate addition and 5 wt % calcium chloride (FIGS. 11 and 12) was possibly a reflection of the marginally higher K_{d} value of potassium compared with sodium citrate.

### Conclusions

Increasing the addition of calcium chloride, calcium nitrate, calcium acetate, sodium hexaphosphate and sodium aluminate from 2 to 5 and 10 wt % significantly increased the injectability of the PCS (p<0.05). However adding sodium citrate above 2 wt % markedly decreased cement injectability.

All of the admixtures added individually at 5 and 10 wt % at a PLR of 4.0 g/ml accelerated the setting of the cement to below 45 minutes, a significant reduction compared with the PC standard (p<0.05). However, decreasing addition of sodium citrate to 2 wt % increased setting times to >6 h. Calcium nitrate and chloride at 5 wt % admixture additions produced the highest compressive strength values. Sodium aluminate, sodium hexaphosphate, sodium citrate and calcium acetate admixtures produced significantly lower compression strength values than the standard PCs (p<0.05). Sodium hexaphosphate, a CPC superplasticiser, has not previously been investigated with PC, while calcium acetate has not been tested above 2.5 wt % additions.

The combination of citrate based admixtures with calcium chloride and nitrate yielded moderate cement extrusion increases compared with using either chloride or nitrate separately. However, the citrate anion caused retardation of the setting reaction of the cements with only combinations of below 0.1 wt % potassium citrate or below 0.05 wt % sodium citrate in combination with 5 wt % calcium chloride setting in under an hour.

The initial aim of the study was to investigate a range of superplasticisers and setting accelerants which would improve the physical characteristics of PC for use in vertebroplasty. Calcium chloride and calcium nitrate were the only two admixtures which both increased cement extrusion, reduced setting time while maintaining similar compressive strength values to unmodified PC. Combining citrate based admixtures with calcium chloride and calcium nitrate moderately increased extrusion but the citrate modified cements could be unfeasible for use in vertebroplasty due to long setting times.

### Example 2

### Cements Containing a Combination of Calcium Chloride and Calcium Nitrate

### Results

### Investigating the Effect of Combining Calcium Chloride and Calcium Nitrate on the Injectability of Portland Cement (PC).

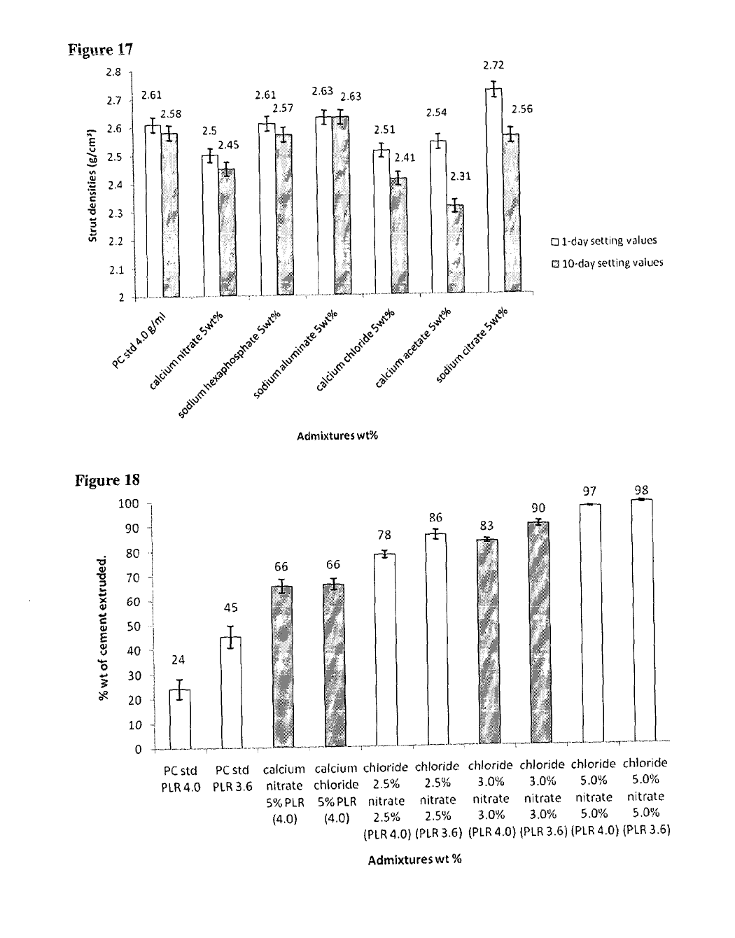

Combining equal 2.5 wt % of calcium chloride and calcium nitrate with Portland cement (PC) increased cement extrusion compared with the individual use of either admixture (FIG. 18). Addition of either 5 wt % calcium chloride or calcium nitrate with PC produced extrusion values of 66 wt %. In comparison, adding equal 2.5 wt % additions of each admixture with PC increased extrusion by over 10%. Further increasing the additions of the combined admixtures increased cement extrusion comparatively with increasing either admixture individually.

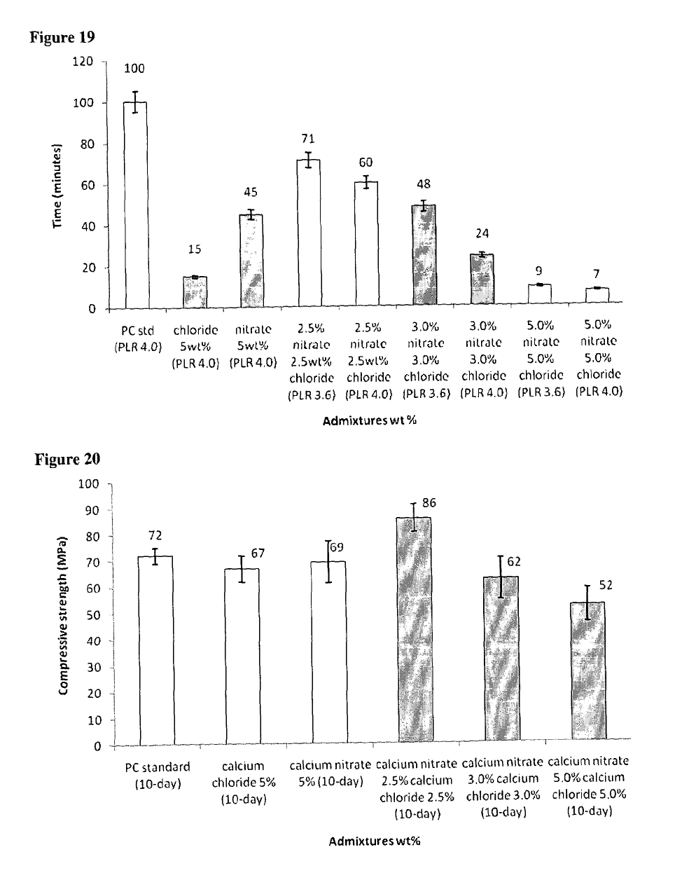

All of the cements containing equal wt % of calcium chloride and calcium nitrate decreased setting times by at least 30% compared with the PC standard (FIG. 19). However, setting times were at least a third slower than using either admixture individually at 5 wt %. Cement setting times decreased with both increasing additions of admixture and increasing PLR. Cements combining 5 wt % of each admixture set in less than 10 minutes.

FIG. 20 illustrates the compressive strength values of the combined admixtures. Cements containing 2.5 wt % of calcium chloride and nitrate produced compressive strength values nearly 20% higher than the PC standard or cements containing individual additions of either 5 wt % calcium chloride or calcium nitrate. In contrast, increasing total admixture addition from 5-10 wt % resulted in a 20% decrease in compressive strength.

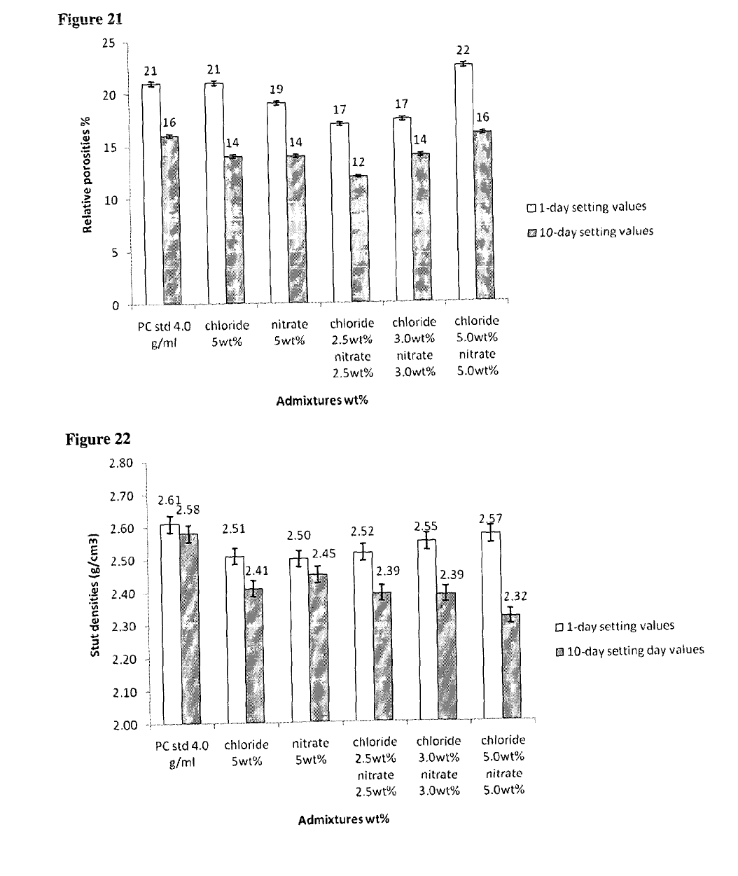

Relative porosities of the cements containing equal quantities of calcium chloride and calcium nitrate were lower than the PC standard up to a total of 6 wt % addition (FIG. 21). Increasing the total wt % of combined admixture to 10 wt % produced comparable relative porosities to the PC standard.

After 1-days setting all of the cements containing combined admixtures possessed similar strut densities to the PC standard (FIG. 22). In contrast, after 10-days setting all of the cements containing combined admixtures produced significantly lower strut densities (p<0.05) than the PC standard.

Elemental Analysis of Portland Cement with Energy Dispersive X-Ray Analysis (EDX).
Elemental Analysis of PC after 1-Day Setting.

Table 4 shows the elemental analysis of the surface of standard Portland cement after 1-days setting. The O/Si ratio indicates that the surface of the cement may be composed of Calcium silicate hydrate (C—S—H). Refer to appendix for original weight % composition of Portland cement 1-days setting.

| Elements | O/Si |
|---|---|
| Observed ratio | 10.9 |
| Suggested compound 1 | C-S-H |
| Theoretical ratio of suggested compound | 3.71 |
| % difference from observed ratio | 66% |

Elemental Analysis of PC after 30-Days Setting.

Table 5 shows the elemental analysis of the surface of standard Portland cement after 30-days setting. The O/Si ratio suggests C—S—H as the main phase in contrast the Ca/Si ratio indicates the presence of tri/di calcium silicates. Refer to appendix for original weight % composition of Portland cement 30-days setting.

| | Ca/O | Ca/Si | O/Si |
|---|---|---|---|
| Observed ratio | 0.85 | 3.79 | 4.4 |

| | calcium | | |
|---|---|---|---|
| | sulphate | tri-calcium | |
| Suggested compound 1 | anhydrous | silicates (alite) | C-S-H |
| Theoretical ratio of | 0.63 | 4.29 | 2.14 |
| suggested compound | | | |
| % difference from | 25.9% | 13.2% | 51% |
| observed ratio | | | |

| | | di-calcium |
|---|---|---|
| Suggested compound 2 | C-S-H | silicates (belite) |
| Theoretical ratio of | 0.58 | 2.86 |
| suggested compound | | |
| % difference from | 25.9% | 24.5% |
| observed ratio | | |

Elemental Analysis of the PCS Containing 5 wt % Sodium Hexaphosphate after 1-Day's Setting.

Table 6 shows elemental analysis of the surface of Portland cement containing 5 wt % sodium hexaphosphate after 1-days setting. The Calcium/Oxygen ratio indicates that the surface of the cement may be composed of calcium silicate hydrate (C—S—H).

| | Ca/O |
|---|---|
| Observed ratio | 0.83 |
| Suggested compound 1 | Calcium sulphate anhydrous |
| Theoretical ratio of suggested | 0.63 |
| compound | |
| % difference from observed ratio | 24.0% |
| Suggested compound 2 | C-AF-H |
| Theoretical ratio of suggested | 0.63 |
| compound | |
| % difference from observed ratio | 24.0% |

Elemental analysis of the PCS containing 5 wt % sodium aluminate after 1-days setting. Table 7 shows the elemental analysis of the surface of Portland cement containing 5 wt % sodium aluminate after 1-days setting. The majority of ratios indicate that monosulphate is present on the surface of the cement calcium sulphate hemihydrates may also be present.

| Elements | Ca/O | Ca/Al | O/Al |
|---|---|---|---|
| Observed ratio | 0.51 | 3.53 | 6.93 |

| | Calcium | | |
|---|---|---|---|
| | sulphate | | |
| Suggested compound 1 | hemihydrate | monosulphate | monosulphate |
| Theoretical ratio of | 0.50 | 2.96 | 6.52 |
| suggested compound | | | |
| % difference from | 0.02% | 16.1% | 6.0% |
| observed ratio | | | |

| | | Calcium |
|---|---|---|
| Suggested compound 2 | monosulphate | aluminoferrite |
| Theoretical ratio of | 0.45 | 2.96 |
| suggested compound | | |
| % difference from | 11.8% | 16.1% |
| observed ratio | | |

### X-Ray Diffraction Analysis

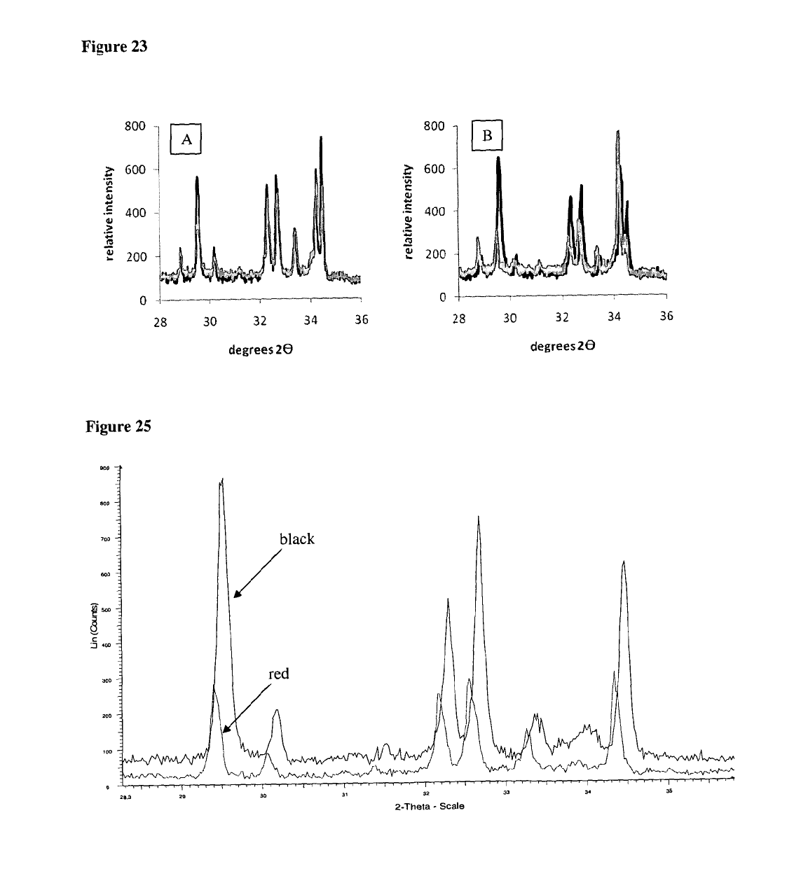

After 1-day setting the intensity of the peaks representing calcium hydroxide at 18, 28-29 and 34° theta were more intense in PC containing 5 wt % calcium chloride than in the PC standard. PC and cements containing 5 wt % calcium chloride also possessed peaks at 9 and 16° theta corresponding to ettringite formation (FIG. 23A).

After 30-days setting PC containing 5 wt % calcium chloride possessed calcium hydroxide peak intensities higher than the PC standard. For both cements there was also a considerable depreciation of the single calcium silicates peaks at 29-30, 34-35°, 41 and 52° theta and the double peak at 32-33° thetas (FIG. 23B).

### Discussion

Combining Calcium Chloride and Calcium Nitrate with the Portland Cement System (PCS)

The hydration of Portland cement is complex, with at least six known hydration reactions occurring simultaneously (Tennis, P., 8th annual concrete conference. 2007: Pennsylvania. p. 1-20). The effect of calcium nitrate has previously been linked to the hydration of the belite (C₂S) phase (Aggoun, S., et al., Construction and Building Materials, 2008. 22(2): p. 106-110). Belite dissolves slowly to form short calcium silicate hydrate (C—S—H) bonds which provide long term cement strength (Chikh, N., et al., Materials and Structures, 2008. 41(1): p. 31-36), the short C—S—H bonds possibly contributed to the high cement strengths for PC containing 5 wt % calcium nitrate (FIG. 20). In contrast, the high strength values obtained with the individual addition of 5 wt % calcium chloride have been previously linked with the production of a more structured, alite, C—S—H bond formation compared with standard PC (Ber, B. S., J. F. Hatton, and G. P. Steward, Journal of Endodontics, 2007. 33(10): p. 1231-1234). Possibly, when these two admixtures were combined in equal 2.5 wt % quantities each admixture may have accelerated a different set of hydration reactions. The combination of the individual accelerating effects may have contributed to the increased compressive strength (FIG. 20) and cement extrusion (FIG. 18) of PC containing both admixtures but conversely extended the setting times (FIG. 19) of the cements compared with using either admixture individually. The relative porosities of the combined cements, FIG. 21, increased with increasing admixture addition. This may have resulted in an increase in the critical flaw size of the cements leading to the decreased compression strengths for cements containing 5 wt % of calcium chloride and nitrate (Eden, N. and J. Bailey, Journal of Materials Science, 1984. 19(1): p. 150-158). In contrast, strut densities of the combined cements, FIG. 22, decreased with increasing addition of admixtures, suggesting that the degree of cement hydration increased with increasing admixture addition. This should have resulted in a strong strut structure for the cement containing the combined admixtures.

### Scanning Electron Microscopy (SEM) and Energy Dispersive X-Ray Analysis (EDX).

Standard PC and PC containing 5 wt % calcium chloride or calcium nitrate all shared similar cement surface morphologies after 1-day setting. The elemental analysis of standard PC suggests that the microcrystalline structure on the cement surface may be composed of C—S—H which is the normal hydration product of alite or belite (Tennis, P., 8th annual concrete conference. 2007: Pennsylvania. p. 1-20). However, the lack of silicon content found during the investigation indicates further X-ray diffraction analysis may be required to ascertain the exact phase composition. EDX analysis suggest that after 30-days setting the structured, ordered surface of the PC standard was composed of C—S—H phase but unreacted belite may also be present (Table 5). As belite reacts slowly 30-days may be insufficient for all the clinker phase to react (Chikh, N., et al., Materials and Structures, 2008. 41(1): p. 31-36).

The 3-20 μm crystals present on the surface of 5 wt % sodium hexaphosphate, may be unreacted calcium sulphate anhydrous present in the clinker phase, or calcium aluminoferrite hydrate the hydration product of tetracalcium aluminoferrite (analysis Table 6). However, lack of a definitive silicon peak prevents a more precise compositional analysis. Again, X-ray diffraction analysis may be required to ascertain the precise phase composition as it analyses the entire surface of the cement not just a single point.

The 5 wt % sodium aluminate cements possessed flat, hexagonal crystals approximately 10 μm in diameter and needle-like crystals over 20 μm in diameter. Both of these crystals have similar morphologies to monosulfate crystals which are produced during flash setting (Baur, I., et al., Cement and Concrete Research, 2004. 34(2): p. 341-348). Flash setting is the early hydration of the tricalcium aluminate phase to form monosulphate and is normally caused by insufficient gypsum in the clinker phase to coat the aluminate particles (normal hydration produces ettringite) (Nonconventional concrete technologies: renewal of the highway infrastructure, in National Research Council, National Materials Advisory Board, Commission of Engineering and Technical Systems. 1997. p. 14-24). An excess of aluminate in the cement paste may have decreased the aluminate/calcium sulphate ratio possibly leading to the monosulphate production.

### X-Ray Diffraction (XRD) Analysis

After 24 h and 30 days cements containing 5 wt % calcium chloride possessed peaks corresponding to crystalline calcium hydroxide (portlandite) which were higher or equal to PC standard. As the above equations are stoichiometric and peak intensities are indicative of quantity this may also suggest an increase in C—S—H production in these cements. The low strut densities of all of these cements also support increased C—S—H production. After 30-days setting both the standard PCs and calcium chloride cements demonstrated a dramatic decrease in calcium silicate levels possibly indicating a conversion to the hydrated C—S—H cement phase.

### Example 3

### Citrate Based Cements

### Results

Characterisation of PC with Various Admixtures with X-Ray Diffraction Analysis (PC Batch 2005)

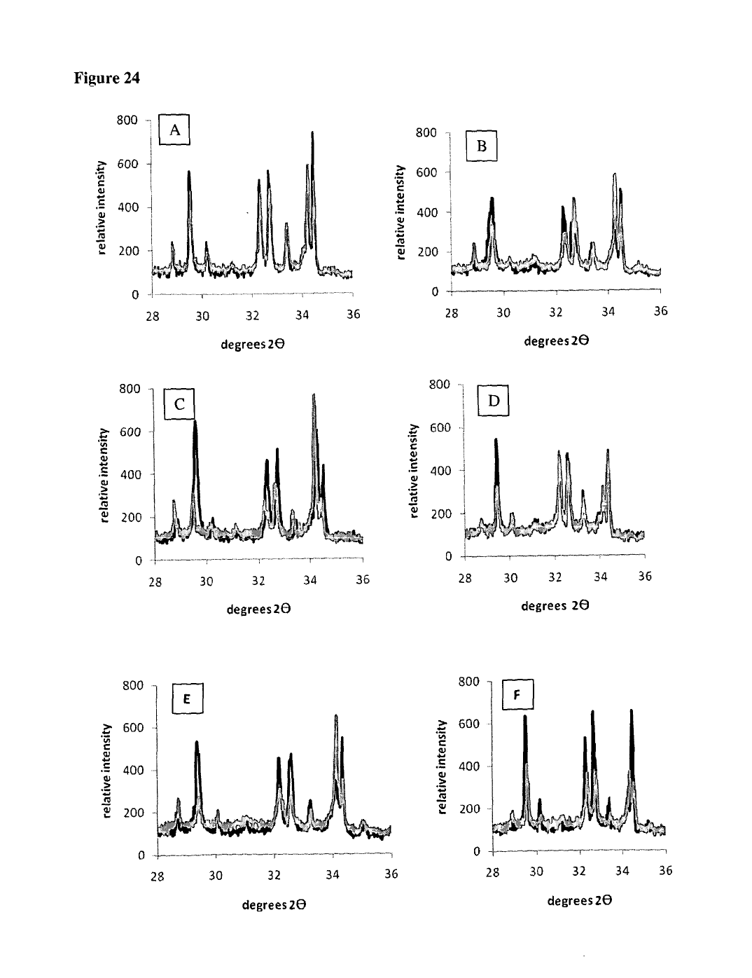

After 1-day setting the intensity of the peaks representing calcium hydroxide at 18, 28-29 and 34° thetas were all more intense in PC containing 5 wt % calcium acetate and calcium nitrate cements than in the PC standard (see FIG. 24). The calcium hydroxide peaks in the calcium chloride cements were comparable with the PC standard. All three of these cements also possessed peaks at 9 and 16° theta corresponding with ettringite formation. In contrast, PC containing sodium hexaphosphate and sodium citrate had no calcium hydroxide or ettringite peaks.

After 30-days setting PC containing 5 wt % calcium acetate, calcium nitrate and calcium chloride all possessed calcium hydroxide peak intensities higher than the PC standard. In all three of these cements there was also a considerable decrease of the single calcium silicates peaks at 29-30, 34-35°, 41 and 52° theta and the double peak at 32-33° theta. After 30-days both the PC containing 5 wt % sodium hexaphosphate and sodium citrate demonstrated a moderate increase in calcium hydroxide peak intensities but these were still lower than the PC standard. These cements also had no discernable decrease in the peaks corresponding with calcium silica.

X-Ray Diffraction Comparison of PC Batches from 2005 and 2010

The overall peak intensities for the 2005 batch of PC were less intense than for the 2010 batch (FIG. 25). The ‘Full-width half peak maximum’ values for the peak at 32.5° theta were 0.140 and 0.114 for the old batch and new batch respectively. Both of these features indicate a general degradation of the old batch of cement. However, lack of formation of any Calcium-silicate-hydrate indicated that neither of the cement powders was degraded by reaction with water.

### Investigating Citrate Based Cements (2010 PC Batch)

### Investigating the Effect of Various Cement Liquefiers on the Injectability of PC

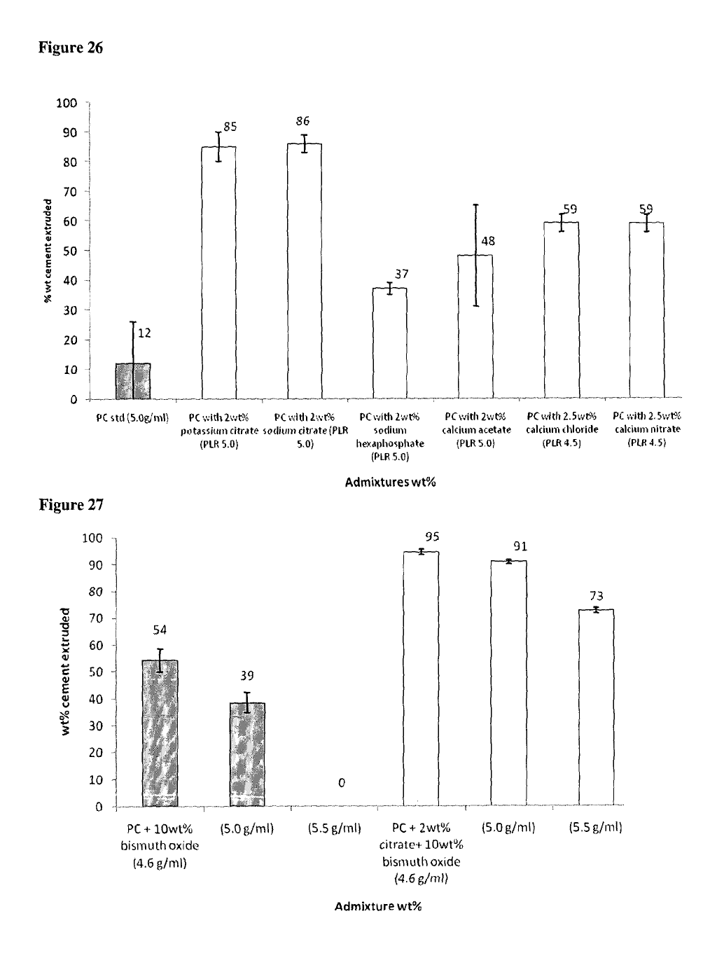

Additions of either sodium or potassium citrate produced over a 600% increase in cement extrusion compared with the PC standard at a powder-to-liquid ratio of 5.0 g/ml (FIG. 26). In contrast, the other liquefiers extruded more than 30% less cement than the citrate based admixtures even with a reduced powder-to-liquid ratio.

The addition of 10 wt % radio-pacifier bismuth oxide to the PC increased injectability from 12 to 39 wt % (FIG. 27). A similar wt % addition of bismuth oxide to PC containing 2 wt % sodium citrate increased cement injectability by 7% compared with the individual use of the citrate admixture.

Investigating the Setting Times of PC with Sodium Citrate and Bismuth Oxide

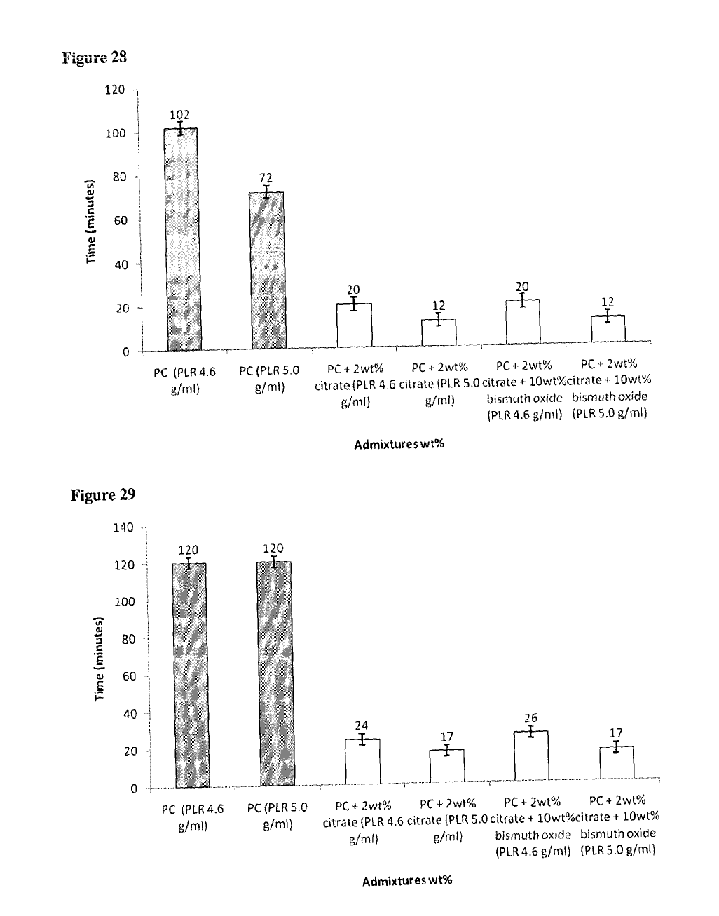

The addition of 2 wt % sodium citrate accelerated the setting time of PC to below 25 minutes (FIG. 28), a significant reduction compared with the PC standard (p<0.001). The final setting times for PCs containing sodium citrate were on average only five minutes slower than the initial setting times (FIG. 29). The incorporation of the radiopacifier bismuth oxide did not significantly increase cement setting times for the citrate based cements (p<0.005).

Investigating the Compressive Strengths and Relative Porosities of PC with Citrate and Bismuth Oxide

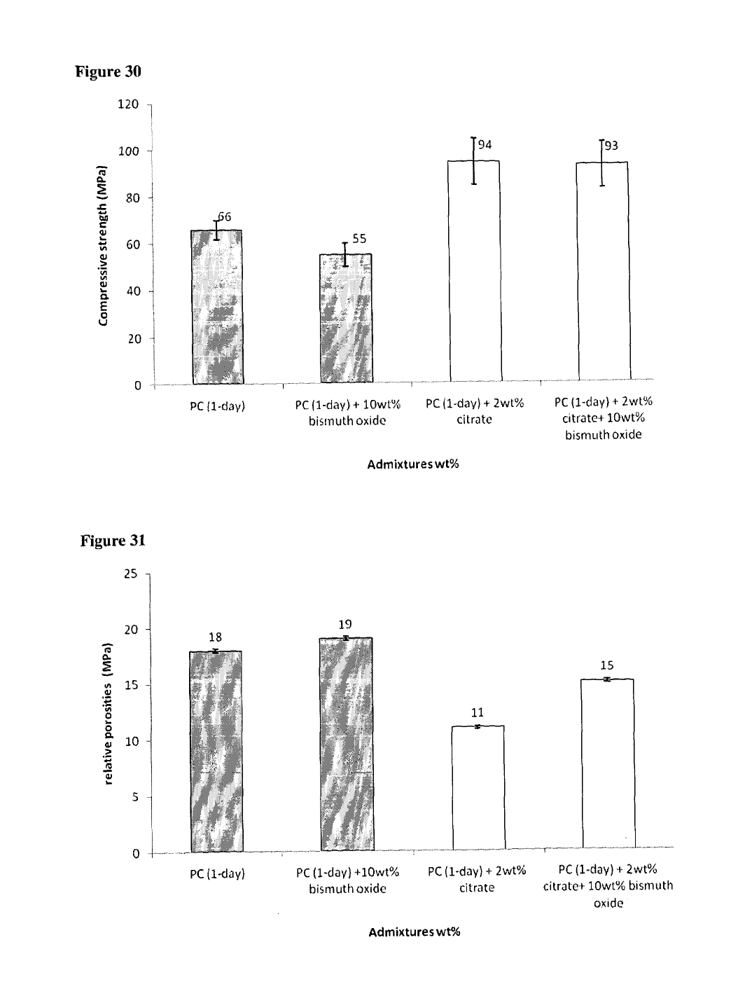

The addition of 2 wt % sodium citrate significantly increased the compressive strength of PC at a PLR of 5.0 g/ml (p<0.001) (FIG. 30). The addition of bismuth oxide to the PC standard significantly reduced the cements compressive strength values (p<0.001). In contrast, bismuth oxide did not significantly affect the compressive strength values for the cement containing sodium citrate (p<0.005).

The citrate based cements all possessed significantly lower relative porosities compared with the PC standard at a PLR of 5 g/ml (FIG. 31). The addition of bismuth oxide did not significantly increase the porosities of the PC standard but did increase the porosity of the 2 wt % citrate cements.

### Scanning Electron Microscopy (SEM) Analysis of PC Containing 2 wt % Sodium Citrate

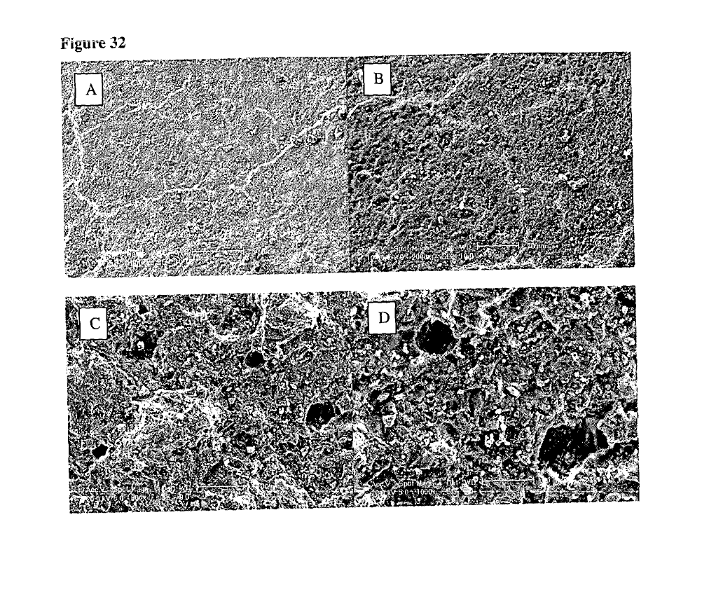

The surface of PC with 2 wt % sodium citrate appeared smooth with very few pores compared with the standard PCs (FIG. 32).

### Cell Culture

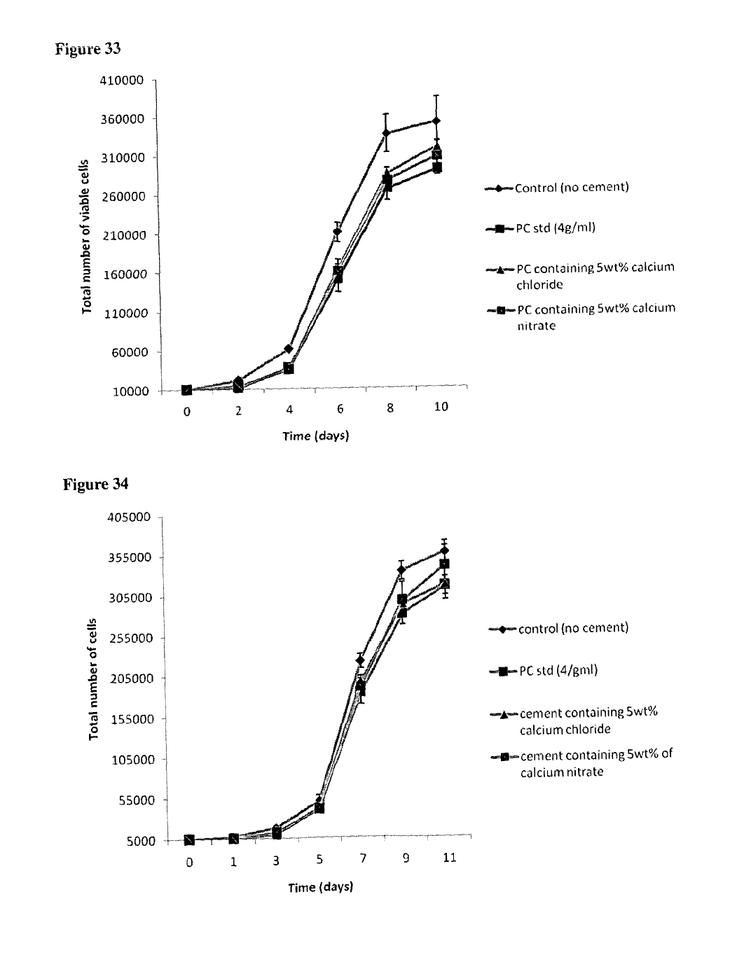

FIG. 33 illustrates the growth curves obtained for 3T3 fibroblast cells (passage 5) (8) incubated with PC standard and PC containing 5 wt % calcium chloride and nitrate. The cells were seeded into a 35 mm Petri-dish at a cell density of 1×10⁴ cells/ml on day 0. The cements were placed into the centre of the Petri-dishes at the same time.

The initial lag phase of the cells was between 0-4 days, during which time the cells adhered to the dishes surface. The cells in the exponential phase, between 4-8 days, doubled approximately twice per day, the growth rate of the control dishes with no cement were significantly higher than dishes containing cements. There was no significant difference in growth rates between any of the cement containing dishes. Maximum cell number in a confluent monolayer appears to be approximately 360,000 cells and was achieved after 10-days of cell growth.

In contrast, in the second experiment, only 5000 3T3 cells (passage 5) were seeded into a 35 mm Petri-dish at a cell density of 5×10³ cells/ml. The cements were placed into the dishes 24 hrs after this. FIG. 34 illustrates the growth curves obtained for these cells.

Again, at the peak of the exponential growth phase the cell population was doubling approximately twice per day. However, unlike the previous experiment there was only a significant difference between the cell growth rates of the control and dishes containing cements after 7-days of growth. The maximum cell number also appeared to be approximately 360,000 which was achieved after 11-days.

### Discussion

### X-Ray Diffraction Analysis for Calcium Chloride, Calcium Nitrate, Sodium Hexaphosphate, Calcium Acetate and Sodium Citrate. (PC Batch 2005)

As the conversion of di- and tri-calcium silicates to C—S—H are stoichiometric equations (eq. 7 & 8), the presence of increased calcium hydroxide in cements containing calcium chloride, calcium nitrate and calcium acetate may have indicated accelerated C—S—H formation (Camilleri, J., F. E. Montesin, and K. Brady, Dental Biomaterials, 2005. 21: p. 297-303). As the hydraulic formation of C—S—H represents the major setting reaction of PC this may explain the observed increased compressive strengths of the chloride and nitrate based cements.

2Ca₂SiO₅+7H₂O->3CaO.2SiO₂.4H₂O+Ca(OH)₂

2Ca₃SiO₅+7H₂O->3CaO.2SiO₂.4H₂O+3Ca(OH)₂ Eq. 7 and 8

In contrast, the low compressive strengths of the 1-day set cements containing 5 wt % sodium citrate and sodium hexaphosphate were mirrored by a lack of calcium hydroxide formation. Interestingly, the increase of strength of the 5 wt % sodium citrate cements after 30-days setting was also reflected by the XRD with increased calcium hydroxide production.

Another wagon for the high strength of the chloride and nitrate cements may be the presence of an ettringite phase in these cements. Hydration of tricalcium aluminate (eq. 9) to ettringite is another important reaction for early cement strength.

CaO.Al₂O₃+3(CaO.SO₃.2H₂O)+26H₂O->6CaO.Al₂O₃.3SO₃.32H₂O Eq. 9

The combined lack of C—S—H and ettringite product formation in the citrate and hexaphosphate cements may explain the observed low compressive strengths.

### X-Ray Diffraction of the 2010 vs 2005 Batches of PC

The decrease in peak intensities for the batch of PC from 2005 may have been an indication of the degradation of the reactant calcium-silicate phase of the cement into compounds which were similar in structure but were unable to react with water. This un-reacted agglomerate possibly led to the slightly higher relative porosity values for the 2005 compared with the 2010 PC batch.

### Effect of 2 wt % Sodium Citrate on the Injectability, Setting Times, Compressive Strengths and Relative Porosities of PC (New Batch of PC Batch 2010)

Heat of hydration experiments have previously indicated that the citrate anion may prevent the dissolution of both the alite and aluminate phases of PC, both of which are essential for cement paste setting (Moschner, G., et al., Cement and Concrete Research, 2009. 39(4): p. 275-282). Prevention of phase dissolution may explain the fluid consistency of the 2 wt % citrate cements which generated cement extrusion values of over 85 wt % (FIG. 34). Singh et al (Singh, N. B., A. K. Singh, and S. Prabha Singh, Cement and Concrete Research, 1986. 16(6): p. 911-920) utilised zeta-potential measurements to investigate the interaction of the citrate anion with the cement constituents. It was found that the drop in positive surface charge density of the set cement was an indication of the negative citrate anion binding to the positive cement surface. It has been suggested that binding results in repulsion between cement particles both electrostatically and sterically due to the size of the citrate anion. This could have dispersed the cement aggregates leading to the superplasticising effect (Erdogdu, S., Cement and Concrete Research, 2000. 30(5): p. 767-773). Both sodium and potassium citrate share similar dissociation constants (Kd) of 0.2M and 0.3M respectively (Mackenzie, W., American Chemistry Journal, 1960. 65: p. 159-161). Therefore, the concentration of either of these two anions in solution will be similar, which may have accounted for the comparable cement extrusion values. Zeta-potential measurements have also indicated that hexaphosphate interacts with the positively charged surface of PC and may have acted through a similar mechanism to citrate (Hesaraki, S., A. Zamanian, and F. Mortarzadeh, Journal of Biomedical Materials Research Part A, 2009. 88A(2): p. 314-321).

For other ceramic cements the addition of sodium citrates significantly increased the compressive strengths and decreased setting times (Gbureck, U., et al., Biomaterials, 2004. 25(11): p. 2187-2195). The strength increase was accompanied by a decrease in relative porosities of the cements that indicated a decrease in either non-consumed dry powder agglomerates or un-reacted water. The citrate based PCs in the present experiments also possessed significantly lower porosities and the cement structure appeared less porous than the PC standard. This suggests a similar decrease in powder agglomerates or un-reacted water in the citrate based cements compared with the PC standard.

### Cell Culture

The similarity between the growth rates of 3T3 cells in both controls and those exposed to PCs indicated that the presence of set PC does not appear to adversely affect the proliferation of the 3T3 fibroblasts. Other authors have also demonstrated bioactivity with set PC. Mitchell (Mitchell, P. J. C., et al., Biomaterials, 1999. 20(2): p. 167-173) demonstrated what appeared to be a confluent monolayer of osteosarcoma cells (MG63) on the surface of MTA. Koh (Koh, E. T., et al., Journal of Biomedical Materials Research, 1997. 37(3): p. 432-439) also reported cell attachment of MG63 cells with MTA. However, on closer inspection of the images presented the cells appear to be more similar in size and shape to bacteria than mammalian cells and some of the images appear to be more structurally similar to cement phases such as C—S—H and ettringite than mammalian cells (Baur, I., et al., Cement and Concrete Research, 2004. 34(2): p. 341-348). Gene expression studies have indicated that PC and MTA induce an osteogenic phenotype in periodontal ligament fibroblasts (PDL) with the increased expression of genes such as osteopontin, osteonidogen and osteonectin (Bonson, S., B. G. Jeansonne, and T. E. Lallier, Journal of Dental Research, 2004. 83(5): p. 408-413).

1. A vertebroplastic cementitious composition, the composition comprising:
a solid phase comprising Portland cement and a biocompatible additive which is a plasticiser and a hardening accelerant, wherein the amount of additive is between about 1% and about 15% by weight relative to the Portland cement; and
an aqueous liquid phase,
wherein the ratio of the solid phase to the liquid phase is between about 3 g/ml and about 6 g/ml.

2. The composition of claim 1, wherein the solid phase comprises at least about 70% by weight Portland cement.

3. The composition of claim 1, wherein the biocompatible additive is selected from one or more of the following: calcium chloride, calcium nitrate, sodium aluminate, sodium hexaphosphate, calcium acetate, citric acid, sodium citrate, calcium citrate and potassium citrate.

4. The composition of claim 1, wherein the biocompatible additive is sodium citrate or potassium citrate.

5. The composition of claim 1, wherein the biocompatible additive is the combination of calcium chloride and calcium nitrate.

6. The composition of claim 1, wherein the amount of additive relative to the Portland cement is between about 4% and about 11% by weight.

7. The composition of claim 1, wherein the amount of additive relative to the Portland cement is between about 1.5% and about 2.5% by weight.

8. The composition of claim 1, wherein the ratio of the solid phase to the liquid phase is between about 4.5 g/ml and about 5.5 g/ml.

9. The composition of claim 1, wherein the ratio of the solid phase to the liquid phase is between about 3.5 g/ml and about 4.5 g/ml.

10. The vertebroplastic cementitious composition of claim 1 comprising:
a solid phase comprising Portland cement, calcium chloride, and calcium nitrate, wherein the amount of each of the calcium chloride and the calcium nitrate is between about 2% and about 3.5% by weight relative to the Portland cement; and
an aqueous liquid phase,
wherein the ratio of the solid phase to the liquid phase is between about 3.5 g/ml and about 4 g/ml.

11. The composition of claim 10, wherein the amount of each of the calcium chloride and the calcium nitrate is between about 2.5% and about 3% by weight relative to the Portland cement.

12. The vertebroplastic cementitious composition of claim 1 comprising:
a solid phase comprising Portland cement and a citrate salt, wherein the amount of citrate salt is between about 1.5% and about 2.5% by weight relative to the Portland cement; and
an aqueous liquid phase,
wherein the ratio of the solid phase to the liquid phase is between about 4.5 g/ml and about 5.5 g/ml.

13. The composition of claim 12, wherein the citrate salt is sodium citrate.

14. The composition of claim 12, wherein the citrate salt is between about 1.8% and about 2.2% by weight relative to the Portland cement and the ratio of the solid phase to the liquid phase is between about 4.8 g/ml and about 5.2 g/ml.

15. The composition of claim 12, wherein the solid phase of the composition further comprises between about 9% and 11% bismuth oxide by weight relative to the Portland cement.

16. A reactive cementitious powder comprising Portland cement and a biocompatible additive which is a plasticiser and a hardening accelerant, wherein the amount of additive is between about 1% and about 15% by weight relative to the Portland cement,

17. The reactive cementitious powder of claim 16 comprising Portland cement, calcium chloride, and calcium nitrate, wherein the amount of each of the calcium chloride and the calcium nitrate is between about 2% and about 3.5% by weight relative to the Portland cement.

18. The reactive cementitious powder of claim 16 comprising Portland cement and a citrate salt, wherein the amount of citrate salt is between about 1.5% and about 2.5% by weight relative to the Portland cement.

19. The reactive cementitious powder of claim 16, wherein the powder comprises at least about 70% by weight Portland cement

20. A method of forming a vertebroplastic cementitious composition, the method comprising:
mixing a solid phase comprising Portland cement and a biocompatible additive which is a plasticiser and a hardening accelerant, wherein the amount of additive is between about 1% and about 15% by weight relative to the Portland cement, with an aqueous liquid phase at a ratio of the solid phase to the liquid phase of between about 3 g/ml and about 6 g/ml to form a cementitious composition.

21. The method of claim 20 comprising:
mixing a solid phase comprising Portland cement, calcium chloride, and calcium nitrate, wherein the amount of each of the calcium chloride and the calcium nitrate is between about 2% and about 3.5% by weight relative to the Portland cement, with an aqueous liquid phase at a ratio of the solid phase to the liquid phase of between about 3.5 g/ml and about 4 g/ml to form a cementitious composition.

22. The method of claim 20 comprising:
mixing a solid phase comprising Portland cement and a citrate salt, wherein the amount of citrate salt is between about 1.5% and about 2.5% by weight relative to the Portland cement, with an aqueous liquid phase at a ratio of the solid phase to the liquid phase of between about 4.5 g/ml and about 5.5 g/ml to form a cementitious composition.

23.-25. (canceled)

26. A method of treatment comprising introducing the vertebroplastic cementitious composition of claim 1 into a bone cavity and allowing the cementitious composition to harden and set.

27. A preformed cementitious implant formed from the cementitious composition of claim 1.

28. A cementitious implant comprising a solid hydrated composition comprising Portland cement and a biocompatible additive which is a plasticiser and a hardening accelerant, wherein the amount of additive is between about 1% and about 15% by weight relative to the Portland cement.

29. A vertebroplastic cementitious composition comprising:
a solid phase comprising Portland cement and a citrate salt, wherein the amount of citrate salt is between about 1.5% and about 2.5% by weight relative to the Portland cement; and
an aqueous liquid phase,
wherein the ratio of the solid phase to the liquid phase is between about 4.5 g/ml and about 5.5 g/ml.

30. The composition of claim 29, wherein the citrate salt is sodium citrate.

31. A vertebroplastic cementitious composition comprising:
a solid phase comprising Portland cement, calcium chloride, and calcium nitrate, wherein the amount of each of the calcium chloride and the calcium nitrate is between about 2% and about 3.5% by weight relative to the Portland cement; and
an aqueous liquid phase,
wherein the ratio of the solid phase to the liquid phase is between about 3.5 g/ml and about 4 g/ml.

32. The composition of claim 31, wherein the amount of each of the calcium chloride and the calcium nitrate is between about 2.5% and about 3% by weight relative to the Portland cement.
